SEQUENCE LISTING (42 sequences)

SEQ ID NO: 1 - DNA, 24 bp, Artificial
  Forward primer for MMP-1 RNA in the human keratinocyte cell line HaCaT.
agatgaaaggtggaccaacaattt

SEQ ID NO: 2 - DNA, 20 bp, Artificial
  Reverse primer for MMP-1 RNA in the human keratinocyte cell line HaCaT.
ccaagagaatggccgagttc

SEQ ID NO: 3 - DNA, 32 bp, Artificial
  Probe for MMP-1 RNA in the human keratinocyte cell line HaCaT.
agagagtacaacttacatcgtgttgcggctca

SEQ ID NO: 4 - DNA, 21 bp, Artificial
  Forward primer for MMP-10 RNA in the human keratinocyte cell line HaCaT.
aacagattttgtgggcaccag

SEQ ID NO: 5 - DNA, 21 bp, Artificial
  Reverse primer for MMP-10 RNA in the human keratinocyte cell line HaCaT.
ttcgcaagatgatgtgaatgg

SEQ ID NO: 6 - DNA, 27 bp, Artificial
  Probe for MMP-10 RNA in the human keratinocyte cell line HaCaT.
aggcagggggaggtccgtagagagact

SEQ ID NO: 7 - DNA, 15 bp, Artificial
  Forward primer for MMP-2 RNA in the human keratinocyte cell line HaCaT.
ccctcgcaagcccaa

SEQ ID NO: 8 - DNA, 22 bp, Artificial
  Reverse primer for MMP-2 RNA in the human keratinocyte cell line HaCaT.
cagatcaggtgtgtagccaatg

SEQ ID NO: 9 - DNA, 28 bp, Artificial
  Probe for MMP-2 RNA in the human keratinocyte cell line HaCaT.
tgggacaagaaccagatcacatacagga

SEQ ID NO: 10 - DNA, 21 bp, Artificial
  Forward primer for MMP-9 RNA in the human keratinocyte cell line HaCaT.
cctgagaaccaatctcaccga

SEQ ID NO: 11 - DNA, 21 bp, Artificial
  Reverse primer for MMP-9 RNA in the human keratinocyte cell line HaCaT.
gccacccgagtgtaaccatag

SEQ ID NO: 12 - DNA, 28 bp, Artificial
  Probe for MMP-9 RNA in the human keratinocyte cell line HaCaT.
aggcagctggcagaggaatacctgtacc

SEQ ID NO: 13 - DNA, 18 bp, Artificial
  Forward primer for TIMP-1 RNA in the human keratinocyte cell line HaCaT.
cacccacagacggccttc

SEQ ID NO: 14 - DNA, 20 bp, Artificial
  Reverse primer for TIMP-1 RNA in the human keratinocyte cell line HaCaT.
ctggtgtccccacgaacttg

SEQ ID NO: 15 - DNA, 24 bp, Artificial
  Probe for TIMP-1 RNA in the human keratinocyte cell line HaCaT.
ccctgatgacgaggtcggaattgc

SEQ ID NO: 16 - DNA, 21 bp, Artificial
  Forward primer for beta-carotene 15, 15′-oxygenase RNA in the human keratinocyte cell line HaCaT.
aggaaagaacagctggagcct

SEQ ID NO: 17 - DNA, 18 bp, Artificial
  Reverse primer for beta-carotene 15, 15′-oxygenase RNA in the human keratinocyte cell line HaCaT.
gttccctgcagccatgct

SEQ ID NO: 18 - DNA, 26 bp, Artificial
  Probe for beta-carotene 15,15′-oxygenase RNA in the human keratinocyte cell line HaCaT.
tgagggccaaagtgacaggcaagatt

SEQ ID NO: 19 - DNA, 23 bp, Artificial
  Forward primer for beta-carotene 9′, 10′-oxygenase RNA in the human keratinocyte cell line HaCaT.
gctcaatggctctctacttcgaa

SEQ ID NO: 20 - DNA, 17 bp, Artificial
  Reverse primer for beta-carotene 9′, 10′-oxygenase RNA in the human keratinocyte cell line HaCaT.
cagcgccatcccatcaa

SEQ ID NO: 21 - DNA, 31 bp, Artificial
  Probe for beta-carotene 9′,10′-oxygenase RNA in the human keratinocyte cell line HaCaT.
cgagtttgggaaggataagtacaatcattgg

SEQ ID NO: 22 - DNA, 22 bp, Artificial
  Forward primer for RAR-alpha RNA in the human keratinocyte cell line HaCaT.
gtcctcaggctaccactatggg

SEQ ID NO: 23 - DNA, 24 bp, Artificial
  Reverse primer for RAR-alpha RNA in the human keratinocyte cell line HaCaT.
tgtacaccatgttcttctggatgc

SEQ ID NO: 24 - DNA, 23 bp, Artificial
  Probe for RAR-alpha RNA in the human keratinocyte cell line HaCaT.
ctgcaagggcttcttccgccgca

SEQ ID NO: 25 - DNA, 24 bp, Artificial
  Forward primer for RAR-beta RNA in the human keratinocyte cell line HaCaT.
aaatcatcagggtaccactatggg

SEQ ID NO: 26 - DNA, 27 bp, Artificial
  Reverse primer for RAR-beta RNA in the human keratinocyte cell line HaCaT.
cggtgacaagtgtaaatcatattcttc

SEQ ID NO: 27 - DNA, 28 bp, Artificial
  Probe for RAR-beta RNA in the human keratinocyte cell line HaCaT.
ctgtgagggatgtaagggctttttccgc

SEQ ID NO: 28 - DNA, 20 bp, Artificial
  Forward primer for RAR-gamma RNA in the human keratinocyte cell line HaCaT.
gttcttctggatgcttcggc

SEQ ID NO: 29 - DNA, 22 bp, Artificial
  Reverse primer for RAR-gamma RNA in the human keratinocyte cell line HaCaT.
gtctacaagccatgcttcgtgt

SEQ ID NO: 30 - DNA, 23 bp, Artificial
  Probe for RAR-gamma RNA in the human keratinocyte cell line HaCaT.
aagaagcccttgcagccttcaca

SEQ ID NO: 31 - DNA, 19 bp, Artificial
  Forward primer for RXR-alpha RNA in the human keratinocyte cell line HaCaT.
aagcacatctgcgccatct

SEQ ID NO: 32 - DNA, 16 bp, Artificial
  Reverse primer for RXR-alpha RNA in the human keratinocyte cell line HaCaT.
tgcacccctcgcagct

SEQ ID NO: 33 - DNA, 25 bp, Artificial
  Probe for RXR-alpha RNA in the human keratinocyte cell line HaCaT.
accgctcctcaggcaagcactatgg

SEQ ID NO: 34 - DNA, 24 bp, Artificial
  Forward primer for RXR-beta RNA in the human keratinocyte cell line HaCaT.
tctggatgatcaggtcatattgct

SEQ ID NO: 35 - DNA, 19 bp, Artificial
  Reverse primer for RXR-beta RNA in the human keratinocyte cell line HaCaT.
tcggtgtgaaaaggaggca

SEQ ID NO: 36 - DNA, 24 bp, Artificial
  Probe for RXR-beta RNA in the human keratinocyte cell line HaCaT.
cgggcaggctggaatgaactcctc

SEQ ID NO: 37 - DNA, 20 bp, Artificial
  Forward primer for RXR-gamma RNA in the human keratinocyte cell line HaCaT.
gcctccaggaatcaacttgg

SEQ ID NO: 38 - DNA, 23 bp, Artificial
  Reverse primer for RXR-gamma RNA in the human keratinocyte cell line HaCaT.
ttgatgtcctctgaactgctgac

SEQ ID NO: 39 - DNA, 28 bp, Artificial
  Probe for RXR-gamma RNA in the human keratinocyte cell line HaCaT.
ccacccagctctcagctaaatgtggtca

SEQ ID NO: 40 - DNA, 20 bp, Artificial
  Forward primer for 18S rRNA in the human keratinocyte cell line HaCaT.
cggctaccacatccaaggaa

SEQ ID NO: 41 - DNA, 18 bp, Artificial
  Reverse primer for 18S rRNA in the human keratinocyte cell line HaCaT.
gctggaattaccgcggct

SEQ ID NO: 42 - DNA, 22 bp, Artificial
  Probe for 18S rRNA in the human keratinocyte cell line HaCaT.
tgctggcaccagacttgccctc

(19) United States
(12) Patent Application Publication
(10) Pub. No.: US 2007/0148106 A1
(43) Pub. Date: Jun. 28, 2007

(54) Treatment, prevention, and modulation of aging of the skin

(76) Inventors: Karin Wertz, Rheinfelden (DE); Regina Goralczyk, Grenzach-Wyhlen (DE)

Correspondence Address: Stephen M. Haracz, Esq.;BRYAN CAVE LLP, 33rd Floor, 1290 Avenue of the Americas, New York, NY, 10104, US

(21) Appl. No.: 11/454,376

(22) Filed: Jun. 15, 2006

Related U.S. Application Data
(60) Provisional application No. 60/691,122, filed on Jun. 15, 2005.

(51) Int. Cl. A61K 8/31

(52) U.S. Cl. 424/59

(57) ABSTRACT

Methods to treat, prevent, and modulate aging of the skin are provided. More particularly, methods are provided for treating or preventing non-light induced skin aging in an organism and for reducing the basal MMP-10 expression in unirradiated cells of an organism and/or reducing the basal MMP-1 RNA transcription and protein translation in unirradiated cells of an organism, which include administering an effective amount of β-carotene, a precursor of β-carotene, a derivative of β-carotene, a salt of β-carotene, or a combination thereof to an organism in need thereof. Also provided are compositions for treating and preventing non-light induced aging of the skin. In addition, methods for modulating UV-A induced RNA transcription and polypeptide translation of a matrix metalloprotease (MMP) and treating or ameliorating UVA-induced photoaging by administering to an organism in need thereof an effective amount of a composition comprising β-carotene, a precursor of β-carotene, a derivative of β-carotene, a salt of β-carotene, or a combination thereof are provided. Compositions for modulating the effect of UVA-induced RNA transcription and polypeptide translation of a matrix metalloprotease (MMP) are also provided.

### FIELD OF THE INVENTION

The present invention relates to methods and compositions for treating, preventing, and/or modulating aging of the skin. More particularly, the present invention relates to methods and compositions for modulating MMP, particularly, MMP-1, -3, and -10, transcription and protein expression in an organism by administering β-carotene, precursor of β-carotene, derivative of β-carotene, salt of β-carotene, or combination thereof to that organism.

### BACKGROUND OF THE INVENTION

Solar light has been implicated in the photoaging process via ultraviolet A (“UVA”) radiation (320 to 400 nm; UVA1 340 nm-400 nm, UVA2 320-340 nm) [1, 2], in addition to ultraviolet B (“UVB”) -mediated skin carcinogenesis [3, 4]. UVA induces reactive oxygen species, including singlet oxygen (“¹O₂”) [5-11]. ¹O₂ in turn can regulate the expression level of a variety of genes, including genes involved in photoaging. ¹O₂-mediated gene induction has been shown for matrix metalloprotease-1 (“MMP-1”) [12, 13], heme oxygenase-1 [14], interleukin-1 (“IL-1”) and -6 (“IL-6”) [15], as well as ICAM-1 [16]. Inhibition or moderation of these molecular events may confer photoprotection on target cells. Due to its excellent ¹O₂-quenching capacity [17-21], β-carotene is a promising agent for the prevention of photoaging. Also, β-cartene accumulates in skin, with generally higher concentrations found in epidermis than in dermis [22]. In humans consuming a diet rich in fruits and vegetables, β-carotene is present in skin at concentrations of about 0.1 to 4 μM [22, 23], and can be further accumulated by supplementation [24]. A photoprotective effect of β-cartene is suggested by several observations. Various organisms, including bacteria, plants, and butterflies, employ β-carotene pigmentation as a means to increase their resistance to damage by irradiation [25]. In erythropoietic protoporphyria (EPP) patients, β-cartene supplementation at high doses (180 mg/d) alleviated the symptoms of photosensitization [26-29], which occurs due to accumulation of endogenous porphyrins. β-Carotene quenches the ¹O₂ formed in the presence of these endogenous porphyrins in UVA-exposed skin [30]. β-Carotene also has a mild sun screen effect (SPF2), if supplemented at a high concentration [26, 31-37]. β-Carotene does not, however, act as an optical filter [38], since its absorption maximum lies outside the UVB/UVA range at around 460 nm.

In addition to its ¹O₂ quenching activity, , β-carotene also represents the most important provitamin A, serving as a precursor for the signaling molecule retinoic acid (“RA”). It is thus conceivable that β-cartene could be locally metabolized to RA, and then act via retinoid pathways. Indeed, β-cartene metabolism to retinol has been shown in cultures of human skin fibroblasts, melanocytes and keratinocytes, which take up β-carotene and increase their intracellular retinol concomitantly [39, 40]. The efficacy of topical tretinoin (all-trans RA) in treating photoaging is well established [41-47]. RA acts by stimulating the proliferation of keratinocytes, while inhibiting terminal keratinocyte differentiation [48-51]. As a result, the thickness of the transit amplifying (TA) keratinocyte layer in the epidermis is increased, leading to a smoother appearance of the skin. Moreover, RA can prevent UV induction of MMP-1 [45], and UV repression of dermal collagen expression [46].

Accordingly, it would be advantageous to provide methods and compositions for treating or preventing skin aging and reduction of basal matrix metalloprotease expression and MMP-1 RNA and protein expression in the cells of an organism susceptible to skin aging.

### SUMMARY OF THE INVENTION

One embodiment of the present invention is a method of treating or preventing non-light induced skin aging in an organism. This method includes administering an effective amount of β-carotene, a precursor of , β-carotene, a derivative of β-carotene, a salt of β-carotene, or a combination thereof to an organism in need thereof.

Another embodiment of the present invention is a composition containing an amount of , β-carotene, a precursor of β-carotene, a derivative of β-carotene, a salt of β-carotene, or a combination thereof effective to treat or prevent non-light induced skin aging.

A further embodiment of the present invention is a method of reducing the basal MMP-10 expression in unirradiated cells of an organism. This method includes administering an effective amount of β-carotene, a precursor of β-carotene, a derivative of β-carotene, a salt of β-carotene, or a combination thereof to the organism in need thereof.

An additional embodiment of the present invention is a method for the reduction of the basal MMP-1 RNA transcription and protein translation in unirradiated cells of an organism including administering an effective amount of β-carotene, a precursor of β-carotene, a derivative of β-carotene, a salt of β-carotene, or a combination thereof to the organism in need thereof.

Another embodiment of the present invention is a method for modulating UV-A induced RNA transcription and polypeptide translation of a matrix metalloprotease (MMP). This method includes administering to an organism in need thereof an effective amount of a composition comprising β-carotene, a precursor of β-carotene, a derivative of β-carotene, a salt of β-carotene, or a combination thereof.

A further embodiment of the present invention is a method of treating or ameliorating UVA-induced photoaging. This method includes administering to an organism in need thereof an effective amount of a composition containing β-carotene, a precursor of β-carotene, a derivative of β-carotene, a salt of β-carotene, or a combination thereof, which is sufficient to ameliorate the UVA-induced photoaging.

An additional embodiment of the present invention is a composition for modulating the effect of UVA-induced RNA transcription and polypeptide translation of a matrix metalloprotease (MMP) containing an effective amount of β-carotene, a precursor of β-carotene, a derivative of β-carotene, a salt of β-carotene, or a combination thereof to modulate the transcription and translation of MMPs induced by exposure to UVA.

### BRIEF DESCRIPTION OF THE DRAWINGS

FIG. 1 shows dose and time dependency of β-carotene (βC) uptake by HaCaT cells. Cells were supplemented with 0.5, 1.5 or 3 μM β-carotene for various time periods. Media were changed daily during the first 3 days. Cellular β-carotene uptake was analyzed by HPLC. Values are means ± standard deviation of an experiment with three replicates per time point and condition.

FIG. 2 shows depletion of cellular β-carotene stores by UVA irradiation. HaCaT cells were supplemented with 0.5, 1.5 or 3 μM β-carotene for 2 days prior to UVA (270 kJ/m²) irradiation. Cellular β-carotene content was analyzed by HPLC. Values are means ± standard deviation from an experiment with three replicates.

FIG. 3 shows the time course of MMP-1 (3a) and MMP-10 (3b) induction by ultraviolet A (“UVA”) irradiation. HaCaT cells were pretreated with 1.5 μM β-carotene for 2 days prior to UVA (270 kJ/m²) irradiation. Gene expression at 1, 2.5, 5, and 18 hours after UVA irradiation was analyzed by quantitative reverse transcriptase-polymerase chain reaction (“QRT-PCR”). Gene regulations by UVA and β-carotene are expressed as fold induction relative to the placebo-treated, non-irradiated controls. The graphs show data from two independent experiments. Error bars indicate standard error.

FIG. 4 shows the effect of β-carotene on UVA-induction of MMP-1 (4a), MMP-3 (4b), MMP-10 (4c), MMP-2 (4d), MMP-9 (4e), and TIMP-1 (4f). HaCaT cells were pretreated with 1.5 μM β-carotene for 2 days prior to UVA (270 kJ/m²) irradiation. Gene expression 5 hours after UVA irradiation was analyzed by QRT-PCR. Gene regulation by UVA and β-cartene is expressed as fold induction relative to the placebo-treated, non-irradiated controls. Values are geometric means ± standard error from three independent experiments for MMP-2, MMP-9, and TIMP-1 and from eight independent experiments for MMP-1, MMP-3, and MMP-10.

FIG. 5 shows the effect of β-cartene on D₂O-enhanced UVA induction of MMP-1 (5a), MMP-3 (5b), and MMP-10 (5c). HaCaT cells were pretreated for 2 days with 0.5, 1.5, or 3 μM β-cartene. The cells were irradiated with UVA (270 kJ m²) either in D₂O-containing PBS or in H₂O-containing PBS, to analyze ¹O₂ (“singlet oxygen”) inducibility of genes. Gene expression five hours after UVA irradiation was analyzed by QRT-PCR. Values are geometric means ± standard error from three independent experiments.

FIG. 6 shows the effect of β-cartene on UVA-induced secretion of MMP-1 (6a) and TIMP-1 (6b) by HaCaT cells. HaCaT cells were supplemented with 0.5, 1.5, or 3 μM β-cartene for 2 days prior to UVA (270 kJ/m²) irradiation. MMP-1 and TIMP-1 secretion 24 hours after irradiation was analyzed by ELISA. Each condition was represented by three replicates in the experiment. Values are means ± standard error.

FIG. 7 shows the effect of , β-carotene on transactivation of an RA-dependent reporter construct. HaCaT cells were transiently transfected with the reporter construct pGL3 (RARE)5 tk luc, containing five DR5-type retinoic acid response elements (“RAREs”). Luciferase activity was determined after 40 hours treatment with β-carotene. Values are means ± standard error from two experiments with four replicates each.

FIG. 8 shows β-Carotene non-significantly induced retinoic acid receptor β (“RARβ”) in a dose-dependent manner. HaCaT cells were pretreated for 2 days with 0.5, 1.5, or 3 μM β-carotene. The cells were irradiated with UVA (270 kJ/m²) either in D₂O-containing PBS or in H₂O-containing PBS, to analyze ¹O₂ inducibility of RARβ. RARβ expression 5 hours after UVA irradiation was analyzed by QRT-PCR. Gene regulation by UVA, D₂O, and β-carotene is expressed as fold induction relative to the placebo-treated, non-irradiated controls. Values are geometric means ± standard error from three independent experiments.

### DETAILED DESCRIPTION OF THE INVENTION

One embodiment of the present invention is a method of treating or preventing non-light induced skin aging in an organism. This method includes administering an effective amount of β-carotene, a precursor of , β-carotene, a derivative of β-carotene, a salt of β-carotene, or a combination thereof to an organism in need thereof.

As used herein, the term “organism in need thereof” means an organism suffering from or susceptible to skin aging, for example, non-light induced skin aging. Preferably, the organism is a mammal, more preferably, a human.

As used herein, the term “effective amount” means the amount of a composition or substance sufficient to produce the desired effect in the organism to which the composition or substance is administered. Preferably, an effective amount of β-carotene is from about 1 milligram to about 30 milligrams per day. More preferably, an effective amount of β-carotene is from about 5 milligrams to about 20 milligrams, even more preferably from about 10 milligrams to about 15 milligrams per day.

Another embodiment of the present invention is a composition containing an amount of β-carotene, a precursor of fβ-carotene, a derivative of β-carotene, a salt of β-carotene, or a combination thereof effective to treat or prevent non-light induced skin aging.

Effective dosage forms, modes of administration, and dosage amounts of β-carotene or compositions containing , β-carotene according to the present invention may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of animal, and like factors well known in the arts of medicine and veterinary medicine. In general, a suitable dose of β-cartene according to the invention will be that amount of the compound, which is the lowest dose effective to produce the desired effect. The effective dose of , β-carotene maybe administered as a single dose or as two, three, four, five, six or more sub-doses, administered separately at appropriate intervals throughout the day.

The β-carotene may be administered in any desired and effective manner: as pharmaceutical compositions for oral ingestion, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Preferably, the β-carotene is administered orally or topically. Further, the β-carotene may be administered in conjunction with other treatments. The β-carotene maybe encapsulated or otherwise protected against gastric or other secretions, if desired.

While it is possible for the β-carotene of the invention to be administered alone, it is preferable to administer the β-carotene as a pharmaceutical formulation (composition). The pharmaceutically acceptable compositions of the invention comprise β-carotene as an active ingredient in admixture with one or more pharmaceutically-acceptable carriers and, optionally, one or more other compounds, drugs, ingredients, and/or materials. Regardless of the route of administration selected, the , β-carotene of the present invention is formulated into pharmaceutically-acceptable dosage forms by conventional methods known to those of skill in the art. See, e.g., Remington's Pharmaceutical Sciences (Mack Publishing Co., Easton, Pa.).

Pharmaceutical carriers are well known in the art (see, e.g., Remington's Pharmaceutical Sciences (Mack Publishing Co., Easton, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen β-carotene dosage form and method of administration can be determined using ordinary skill in the art.

The pharmaceutically acceptable compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen β-carotene dosage form and method of administration may be determined using ordinary skill in the art.

Pharmaceutical formulations suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Dosage forms for the topical or transdermal administration include powders, sprays, ointments, pastes, creams, lotions, gels, solutions, patches, drops and inhalants. The active compound may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise β-cartene in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug, it is desirable to slow its absorption from subcutaneous or intramuscular injection. This may be accomplished by the use of a liquid suspension of crystalline or amorphous material having poor water solubility.

The rate of absorption of the drug then depends upon its rate of dissolution which, in turn, may depend upon crystal size and crystalline form. Alternatively, delayed absorption of a parenterally-administered drug may be accomplished by dissolving or suspending the drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

In the present invention, the, β-carotene may be incorporated into various finished products, such as for example, a food, fortified food, functional food, food additive, clinical nutrition formulation, feed, fortified feed, functional feed, feed additive, beverage, dietary supplement, personal care product, nutraceutical, lotion, cream, spray, etc.

A further embodiment of the present invention is a method of reducing the basal MMP-10 expression in unirradiated cells of an organism. This method includes administering an effective amount of β-carotene, a precursor of β-carotene, a derivative of , β-carotene, a salt of β-carotene, or a combination thereof to the organism in need thereof. In the present embodiment, the organisms, amounts of β-carotene, a precursor of β-carotene, a derivative of β-carotene, a salt of β-carotene, or a combination thereof, delivery routes, and composition forms are as defined above.

An additional embodiment of the present invention is a method for the reduction of the basal MMP-1 RNA transcription and protein translation in unirradiated cells of an organism. This method includes administering an effective amount of β-carotene, a precursor of β-carotene, a derivative of β-carotene, a salt of β-carotene, or a combination thereof to the organism in need thereof. In the present embodiment, the organisms, amounts of β-carotene, a precursor of β-carotene, a derivative of β-carotene, a salt of β-carotene, or a combination thereof, delivery routes, and composition forms are as defined above.

Another embodiment of the present invention is a method for modulating UV-A induced RNA transcription and polypeptide translation of a matrix metalloprotease (MMP). This method includes administering to an organism in need thereof an effective amount of a composition comprising β-carotene, a precursor of β-carotene, a derivative of , β-carotene, a salt of β-carotene, or a combination thereof.

As used herein, the term “modulation” means a reduction in the MMP RNA or protein levels compared to an organism to which the β-carotene composition is not administered.

Preferably, the MMP is selected from the group consisting of MMP-1, MMP-3, MMP-10, and combinations thereof. More preferably, the MMP is MMP-1 and MMP-10.

In the present embodiment, the organisms, amounts of β-carotene, a precursor of β-carotene, a derivative of β-carotene, a salt of β-carotene, or a combination thereof, delivery routes, and composition forms are as defined above.

A further embodiment of the present invention is a method of treating or ameliorating UVA-induced photoaging. This method includes administering to an organism in need thereof an effective amount of a composition containing β-carotene, a precursor of β-carotene, a derivative of β-carotene, a salt of β-carotene, or a combination thereof, which is sufficient to ameliorate the UVA-induced photoaging.

Preferably, the effective amount of β-carotene, a precursor of β-carotene, a derivative of β-carotene, a salt of β-carotene, or a combination thereof is sufficient to reduce the level of MMP RNA transcripts and protein in the skin cells of the organism compared to an organism to which the β-cartene composition is not administered. Preferably, the MMP is selected from the group consisting of MMP-1, MMP-3, MMP-10, and combinations thereof. More preferably, the MMP is MMP-1 and MMP-10.

In the present embodiment, the organisms, amounts of β-carotene, a precursor of β-carotene, a derivative of β-carotene, a salt of β-carotene, or a combination thereof, delivery routes, and composition forms are as defined above.

An additional embodiment of the present invention is a composition for modulating the effect of UVA-induced RNA transcription and polypeptide translation of a matrix metalloprotease containing an effective amount of β-carotene, a precursor of β-carotene, a derivative of β-carotene, a salt of β-carotene, or a combination thereof to modulate the transcription and translation of MMPs induced by exposure to UVA.

In the present embodiment, the organisms, amounts of β-carotene, a precursor of , β-carotene, a derivative of β-carotene, a salt of β-carotene, or a combination thereof, delivery routes, and composition forms are as defined above.

The following examples are provided to further illustrate the compositions and methods of the present invention. These examples are illustrative only and are not intended to limit the scope of the invention in any way.

### EXAMPLES

Summary

UVA exposure causes skin photoaging by ¹O₂-mediated induction of e.g. matrix metalloproteases. We assessed whether pretreatment with β-cartene, a ¹O₂ quencher and retinoic acid (RA) precursor, interferes with UVA-induced gene regulation. HaCaT keratinocytes were precultured with β-cartene at physiological concentrations (0.5, 1.5 and 3.0 μM) prior to UVA exposure from a Hönle solar simulator (270 kJ/m²). HaCaT cells accumulated β-carotene in a time and dose-dependent manner. UVA irradiation massively reduced the cellular β-cartene contents. β-Carotene suppressed UVA-induction of MMP-1, MMP-3, and MMP-10, three major matrix metalloproteases involved in photoaging. We show that not only MMP-1, but also MMP-10 regulation involves ¹O₂-dependent mechanisms. β-Carotene dose-dependently quenched ¹O₂-mediated induction of MMP-1 and MMP-10. Thus, like in chemical solvent systems, β-carotene quenches ¹O₂ also in living cells. Vitamin E did not cooperate with β-carotene to further inhibit MMP induction. HaCaT cells produced weak retinoid activity from β-carotene, as demonstrated by mild upregulation of RARβ and activation of an RARE-dependent reporter gene. β-Carotene did not regulate the genes encoding other RARs, retinoid receptors (“RXR”), or the two β-carotene cleavage enzymes. These results demonstrate that β-carotene is photoprotective, and that this effect is mediated by ¹O₂ quenching.

Materials and Methods

Cell Culture

HaCaT cells were obtained from Prof. Fusenig, German Cancer Research Centre, Heidelberg [64]. To gather cells more representative for the upper epidermal layer, we cloned the original cells by endpoint dilution. A subclone was selected, which had a polygonal epithelial morphology and exhibited the highest differentiation capacity. The clone expressed cytokeratins 1 and 10 starting from day 3 in culture, as detected by Western blotting. Cytokeratins were detected using anti-cytokeratin clones AE1/AE3 (Boehringer Mannheim, Germany) and anti-cytokeratin 1, 10 antibody (Biogenesis Ltd., Poole, UK), respectively. Moreover, this clone expressed cytokeratins 1 and 10, as well as involucrin on the RNA level at 3 days post seeding (Wertz et al., unpublished observations). The doubling time of the clone was 16 hours and identical to the parent cell line. Cells were propagated in FAD medium (DMEM/HAM's F12 3:1, Invitrogen); 5% NuSerum IV culture supplement and Mito™ 1:1000 (both Becton Dickinson, Bedford, Mass., USA). On day 0 of the experiment, cells were seeded at 2×10⁵ cells per 60 mm dish. Cells were counted using a Coulter Multisizer (IG Instrumenten Gesellschaft, Zürich, Switzerland). The accuracy of cell counting was approximately 99%. On days 1 and 2, the media were replaced with fresh β-cartene-containing FAD medium without phenol red; 2% NuSerum; penicillin/streptomycin.

Preparation of β-Carotene-Containing Medium

β-Carotene stock solutions and β-carotene-containing media were prepared under reduced light conditions. All-E-β-carotene was synthesized by DSM Nutritional Products (Kaiseraugst, Switzerland). β-Carotene was dissolved in tetrahydrofurane (THF containing 0.025% butyl hydroxytoluol; Fluka Chemie AG, Buchs, Switzerland). Immediately before preparing the 13β-carotene stock solution, THF was purified over a basic aluminum oxide grade 1 (Camag, Muttenz, Switzerland) column. The β-carotene stock solution was prepared fresh for each experiment, and stored under argon at −20° C. until use. To prepare β-carotene-containing medium, the β-carotene stock solution was first diluted 1:1 with ethanol. This β-carotene/solvent mixture was then added to the cell culture medium to give a final concentration of 0.5, 1.5, or 3 μM β-carotene. β-carotene-containing medium was prepared fresh for the daily medium changes. The solvent concentration in the medium was kept constant at 0.5% for all treatment conditions. In previous experiments, it had been verified that the solvent at this concentration is not toxic for HaCaT cells.

Preparation of Vitamin E-Containing Medium

Vitamin E (RRR-α-tocopherol; DSM Nutritional Products, Kaiseraugst, Switzerland) stock solutions and vitamin E-containing media were prepared as described for β-carotene-containing solutions, except that vitamin E was dissolved in ethanol. In experiments addressing the vitamin E effect, vitamin E was used in a final concentration of 50 μM. Again, the solvent concentration was kept constant at 0.03% ethanol for all conditions.

UVA/Simulated Solar Radiation (SSR) Exposure

On day 3 of the experiment, cells were washed six times with Ca/Mg-free PBS containing 2% BSA, and then irradiated with light with a Hönle sun lamp Sol 500 (Dr. Hönle, Plannegg, Germany) at a dose of 270 kJ/m² in Ca/Mg-free PBS (2 hour exposure time at 3.77 mW/m²). The experimental schedule was chosen because the cells had optimal UVA sensitivity after 48 hours in culture. At that time, the cultures had a confluency of about 95%. Confluent cultures were much less sensitive to irradiation.

The spectrum of the Hönle lamp simulates natural sun light with the majority of the spectrum between 320 and 750 nm. The minor UVB component was further reduced to 0.7 W/m² by placing a glass plate adjacent to the metal-halogenide light source. Thus, the light contained mainly the UVA 1 and 2 and visible light fraction. The dose calculation was based on the UVA measurement. Since an effect of visible light on human skin requires higher doses [65] of 1260 kJ/m², we refer to the major active light spectrum as UVA. Pilot experiments with increasing doses of UVA ranging from 50 to 300 kJ/m² showed a maximum response of HaCaT cells with 270 to 300 kJ/m² with respect to MMP-1 induction.

After irradiation, the cells were supplied with fresh β-carotene-containing serum-free medium and kept in the incubator at 37° C., 5% CO₂ until harvest of the samples. In experiments, in which the half-life of ¹O₂ was prolonged by D₂O to enhance its effect [18], PBS was prepared in D₂O, instead of in H₂O, as it was done for the standard conditions described above. Cells were washed twice in D₂O-PBS prior to irradiation in D₂O-PBS. After irradiation, cells were maintained with fresh β-carotene-containing serum-free medium. Sham controls were treated in an identical manner, by placing them under the solar simulator but shielded from light.

HPLC Analysis of Cell Culture Media and Cell Cultures

All extraction and analytical procedures were carried out in brown glass, and under reduced light conditions. Acetonitrile, tert-butylmethylether, acetone and ethanol p. a. were from Merck (Darmstadt, Germany). Ammonium acetate p. a., butylated hydroxy toluene p. a., tetrahydrofuran p. a., triethylamine p. a., were from Fluka Chemie AG (Buchs, Switzerland). HPLC grade solvents for stock solutions, dilutions, and sample solvent mixtures were additionally purified over basic aluminum oxide grade 1 (Camag, Muttenz, Switzerland). To determine the β-carotene content of cells, the cell layer was washed 5 times with PBS/2% BSA. The cells were detached by trypsin/EDTA 0.05/0.02% and centrifuged at 10000×g for 1 min. Cell pellets were lysed with acetone containing 0.025% BHT (v/w), vortex-mixed and dried in a speed-vac. The dried residue was extracted with ethanol/tBME/THF, 9:5:1, containing 0.025% of BHT (v/w) by vigorous vortex-mixing for one minute, and centrifugation for 3 minutes at 10000×g. An aliquot of the clear supernatant was injected into the HPLC system. Cell culture medium was directly extracted with the solvent mixture described above, and the extract was treated as described for the cell pellets.

The HPLC system consisted of a 520 pump, a 565 autosampler cooled at 6° C., a 540+ diode array detector, a SDU 2003 solvent degasser unit and the Chroma 3000 data analysis system from Bio-Tek Instruments (Basel, Switzerland). A Vydac 218TP54 column (250×4.5 mm i.d., 300 angstrom pore wide) from the Separation Group (Hesperia, USA) was used for separation. The mobile phase consisted of acetonitrile/tert.-butylmethylether/aqueous ammonium acetate 80 mM/triethylamine, 73:20:7:0.05, (v/v/v/v) eluted under isocratic condition. The flow rate was adjusted to 1.5 ml/min and the injected sample volume was 25 μl. The effluent was monitored at 325 nm for retinol and retinyl palmitate, 450 nm for β-carotene, and scanned between 190 and 500 nm by the DAD to detect β-carotene-isomers and apocarotenals. Standard solutions from DSM Nutritional Products (Kaiseraugst, Switzerland) in the range of expected sample concentration were used to quantify all-E-β-carotene, (9Z)-β-carotene, (13Z)-β-carotene, 4′-β-apocarotenal, 8′-β-apocarotenal, 12′-β-apocarotenal, all-E-retinol, and retinyl palmitate in cell culture extracts. From the HPLC chromatograms of the standards an average value of the relevant peak areas was divided by the corresponding photometrically measured concentration in a defined injection volume. This resulted in specific HPLC response factors (RF values) for each compound at defined chromatographic conditions. Limits of β-carotene and apocarotenals quantification (LOQ) were in the range of 0.05 to 0.1 μmol.L⁻¹ for media and 0.6 to 1.0 pmol for 1×10⁶ cells. The limit of detection for retinol and retinyl palmitate was below 0.5 pmol for 1×10⁶ cells.

The identification of the major β-carotene metabolites formed in cells was based on expected elution order as well as on absorption spectra obtained by photodiode array detection. To confirm these results, some cell extracts were analysed by APCI⁺ tandem mass spectrometry. The main metabolites formed were identified as (13Z)-β-carotene (m/z: 536), 4′-β-apocarotenal (m/z : 482), 8′-β-apocarotenal (m/z: 416) and monoepoxy-β-carotene (m/z: 620). In addition, a number of minor yet unresolved peaks were detected between 360 and 450 nm. Since the expected amount of RA was below the limit of detection, we used an RARE-driven reporter construct to indirectly measure retinoid activity (see below).

RNA isolation and Quantitative RT-PCR (QRT-PCR)

Total RNA was isolated by using Trizol™ (Invitrogen, Basel, Switzerland) according to the instructions of the manufacturer. Random-primed cDNA was synthesized using the Superscript pre-amplification system for first strand cDNA synthesis (Invitrogen).

cDNA corresponding to 10 ng total RNA was used as template to quantify the relative RNA expression of the genes of interest by TaqMan® real time PCR. The sequences of the primers and probes are shown in Table 1.

TABLE 1
Primers and probes used for QRT-PCR.

| | | | | Basal Expression |
|---|---|---|---|---|
| Transcript | Forward Primer | Reverse Primer | Probe | Level (δCT ± SE) |
| MMP-1 | AGATGAAAGGTGGACCAACA | CCAAGAGAATGGCCGAGTTC | AGAGAGTACAACTTACATCGT | 12.67 ± 0.57 |
| | ATTT | (SEQ ID NO: 2) | GTTGCGGCTCA | |
| | (SEQ ID NO: 1) | | (SEQ ID NO: 3) | |

| | | |
|---|---|---|
| MMP-3 | Hs00233962_m1 Assay-on-Demand (Applied Biosystems) | 22.80 ± 0.70 |

| | | | | |
|---|---|---|---|---|
| MMP-10 | AACAGATTTTGTGGGCACCA | TTCGCAAGATGATGTGAATGG | AGGCAGGGGGAGGTCCGTAG | 15.35 ± 0.569 |
| | G | (SEQ ID NO: 5) | AGAGACT | |
| | (SEQ ID NO: 4) | | (SEQ ID NO: 6) | |
| MMP-2 | CCCTCGCAAGCCCAA | CAGATCAGGTGTGTAGCCAATG | TGGGACAAGAACCAGATCAC | 18.64 ± 1.35 |
| | (SEQ ID NO: 7) | (SEQ ID NO: 8) | ATACAGGA | |
| | | | (SEQ ID NO: 9) | |
| MMP-9 | CCTGAGAACCAATCTCACCG | GCCACCCGAGTGTAACCATAG | AGGCAGCTGGCAGAGGAATA | 21.08 ± 0.89 |
| | A | (SEQ ID NO: 11) | CCTGTACC | |
| | (SEQ ID NO: 10) | | (SEQ ID NO: 12) | |
| TIMP-1 | CACCCACAGACGGCCTTC | CTGGTGTCCCCACGAACTTG | CCCTGATGACGAGGTCGGAA | 5.90 ± 2.40 |
| | (SEQ ID NO: 13) | (SEQ ID NO: 14) | TTGC | |
| | | | (SEQ ID NO: 15) | |
| β-carotene | AGGAAAGAACAGCTGGAGC | GTTCCCTGCAGCCATGCT | TGAGGGCCAAAGTGACAGGC | 23.76 ± 1.27 |
| 15,15′- | CT | (SEQ ID NO: 17) | AAGATT | |
| oxygenase | (SEQ ID NO: 16) | | (SEQ ID NO: 18) | |
| β-carotene | GCTCAATGGCTCTCTACTTC | CAGCGCCATCCCATCAA | CGAGTTTGGGAAGGATAAGT | 19.28 ± 0.469 |
| 9′,10′- | GAA | (SEQ ID NO: 20) | ACAATCATTGG | |
| oxygenase | (SEQ ID NO: 19) | | (SEQ ID NO: 21) | |
| RARα, | GTCCTCAGGCTACCACTATG | TGTACACCATGTTCTTCTGGAT | CTGCAAGGGCTTCTTCCGCC | 9.81 ± 1.29 |
| | GG | GC | GCA | |
| | (SEQ ID NO: 22) | (SEQ ID NO: 23) | (SEQ ID NO: 24) | |
| RARβ | AAATCATCAGGGTACCACTA | CGGTGACAAGTGTAAATCATAT | CTGTGAGGGATGTAAGGGCT | 14.49 ± 0.44 |
| | TGGG | TCTTC | TTTTCCGC | |
| | (SEQ ID NO: 25) | (SEQ ID NO: 26) | (SEQ ID NO: 27) | |
| RARγ | GTTCTTCTGGATGCTTCGGC | GTCTACAAGCCATGCTTCGTGT | AAGAAGCCCTTGCAGCCTTC | 13.20 ± 0.46 |
| | (SEQ ID NO: 28) | (SEQ ID NO: 29) | ACA | |
| | | | (SEQ ID NO: 30) | |
| RXRα | AAGCACATCTGCGCCATCT | TGCACCCCTCGCAGCT | ACCGCTCCTCAGGCAAGCAC | 9.73 ± 0.71 |
| | (SEQ ID NO: 31) | (SEQ ID NO: 32) | TATGG | |
| | | | (SEQ ID NO: 33) | |
| RXRβ | TCTGGATGATCAGGTCATAT | TCGGTGTGAAAAGGAGGCA | CGGGCAGGCTGGAATGAACT | 12.97 ± 0.486 |
| | TGCT | (SEQ ID NO: 35) | CCTC | |
| | (SEQ ID NO: 34) | | (SEQ ID NO: 36) | |
| RXRγ | GCCTCCAGGAATCAACTTGG | TTGATGTCCTCTGAACTGCTGA | CCACCCAGCTCTCAGCTAAAT | 17.90 ± 1.53 |
| | (SEQ ID NO: 37) | C | GTGGTCA | |
| | | (SEQ ID NO: 38) | (SEQ ID NO: 39) | |
| 18S rRNA | CGGCTACCACATCCAAGGAA | GCTGGAATTACCGCGGCT | TGCTGGCACCAGACTTGCCC | 0 (baseline) |
| | (SEQ ID NO: 40) | (SEQ ID NO: 41) | TC | |
| | | | (SEQ ID NO: 42) | |

The PCR analyses were carried out in triplicate and in a multiplex setup, using 18S rRNA as a calibrator gene. The rRNA primers were used at a final concentration of 50 nM, the probe at 100 nM. For quantification of the genes of interest, the primer concentrations were optimized for sensitivity of template detection. Moreover, it was verified that the amplification of the calibrator gene did not interfere with the detection of the gene of interest. PCR reactions were carried out for 40 cycles of 95° C. for 15 sec and 60° C. for 1 min in an ABI7700 (Applied Biosystems, Rotkreuz, Switzerland). Regulation of gene expression was calculated as described in the user bulletin #2 provided by the manufacturer. A threshold cycle (“CT”) is the first PCR cycle in which an amplification signal is detected. Expression levels are given as δCT values. The δCT value describes the level of gene expression as the average PCR cycle, in which the gene of interest was detected first (CT value), subtracted by the CT value of the calibrator gene 18S rRNA (CT_{gene of interest}−CT_{calibrating gene}). 18S rRNA served as a measure for the amount of template in the reaction. Routinely, 18S rRNA was detected between PCR cycle 12 and 13. (SE=standard error.) A low δCT corresponds to a high mRNA level.

Treatment-induced gene regulations are given as fold change relative to the placebo-treated controls. Transcripts were classified as low abundant, if the δCT value was below 23. This expression level is the approximate limit of quantification of the method. Transcripts were called ‘medium abundant’, if their δCT was between 23 and 13. Transcripts detected earlier than at a δCT of 13 were categorized as high abundance transcripts.

ELISA

Release of MMP-1, and TIMP-1 into cell culture supematant was determined by ELISA at 24 hours after irradiation. MMP-1 and TIMP-1 release was measured using MMP-1 and TIMP ELISA from CALBIOCHEM (San Diego, Calif., USA). The ELISAs were performed according to the manufacturers' instructions.

Reporter Gene Assay

HaCaT cells were seeded at a density of 3×10⁶ cells/well in 6 well plates (BD Biosciences, Basel, Switzerland) in FAD medium containing 2% NuSerum. The cells were transfected the next day using 1 μg reporter plasmid (pGL3 (RARE)5 tk luc) and 5 μl Lipofectin (1 μg/μL; Invitrogen, Basel, Switzerland) per well. The RARE is a DR5 element identical to the wild type element of the RARβ2 promoter. The spacing between the DR5 sites is 25 nucleotides. The transfections were performed for 7.5 hours in serum-free FAD medium according to the manufacturer's protocol. The transfections were stopped by replacing the media with FAD/2% NuSerum, or FAD/2% NU serum containing 1 or 3 μM β-carotene, respectively. The solvent concentration was 0.5% THF/ethanol (1:1) for all media. The cells received fresh media the next day. Transactivation of the reporter gene was determined after 40 hours of β-carotene treatment.

To generate the RA standard curve, 9-cis RA and all-trans RA were used together at concentrations ranging from 10⁻¹⁰ to 10⁻⁸ M each. 40 hours later, the cells were washed 6 times with PBS/2% BSA. Cells were irradiated as described above. To prepare cell extracts, the cells were detached from the culture dishes by trypsinisation, and washed in PBS. The cell pellets were dissolved in 500 μl 0.1 M KHPO₄, and the cells were disrupted by three freeze/thaw cycles. Relative luciferase units (RLU) were quantified in a luminoskan reader (Thermo Labsystems, Vantaa, Finland), and corrected by protein concentrations as determined with the BCA assay (Pierce, Rockford, USA).

Statistical Analysis

The results were analyzed for significant treatment effects by ANOVA. If the ANOVA returned a P value below 0.05, the treatment effect was considered significant. Only significant effects were further analyzed by the post-hoc test Fisher's PLSD test, to allow for multiple pair-wise comparisons of treatment conditions, and to detect dose-dependent effects. Again, effects with a P value below 0.05 were regarded as significant. The statistical analysis was done using the software package Statview (SAS Institute Inc., Cary, USA).

Results

Time- and Dose-Dependent Accumulation of β-Carotene in HaCaT Cells

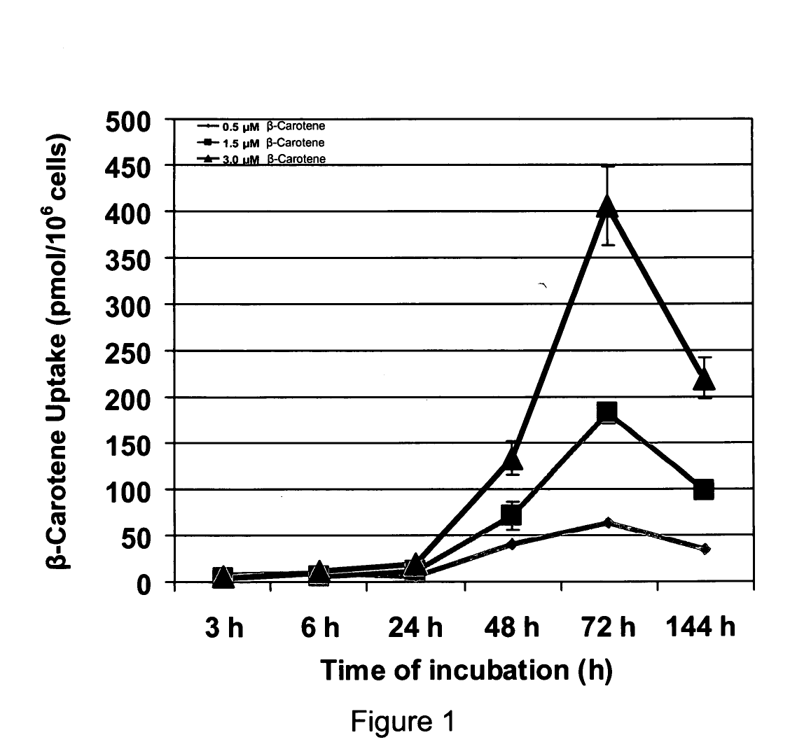

HaCaT cells were supplemented with β-carotene-containing medium for 3, 6, 24, 48, 72, or 144 hours and subsequently analyzed for their β-carotene content by HPLC analysis. β-Carotene was time-dependently accumulated in HaCaT cells, with the peak β-carotene concentration being achieved after 72 hours of supplementation (FIG. 1). After this time point, when the cells were kept for an additional 3 days without adding fresh β-carotene-containing media, the β-carotene contents dropped to about half the concentration observed at 72 h. The β-carotene concentration in cells was dose-dependent, and increased from 63 pmol/million cells at 0.5 μM to 406 pmol/million cells at 3.0 μM within a culture period of 72 h.

UVA Irradiation Lead to Depletion of the Cellular β-Carotene Content

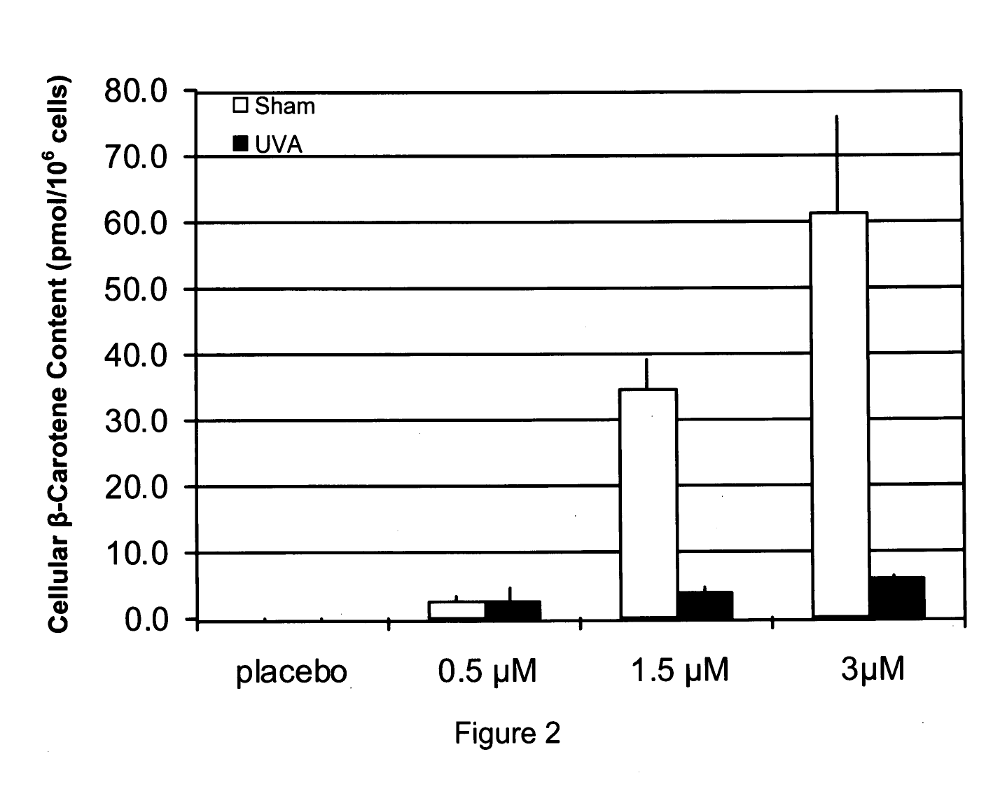

Cells supplemented with 0.5, 1.5, or 3 μM β-carotene for 2 d, were irradiated with 270 kJ/m² UVA, to determine the effect of irradiation on the β-carotene content of the cells. UVA irradiation diminished the β-carotene stores to about 13% in cells incubated in 1.5 or 3 μM β-carotene (FIG. 2). UVA did not reduce the cellular β-carotene content after incubation with 0.5 μM β-carotene.

β-Carotene Reduced UVA-Induced MMP-1, MMP-3, and MMP-10 Induction

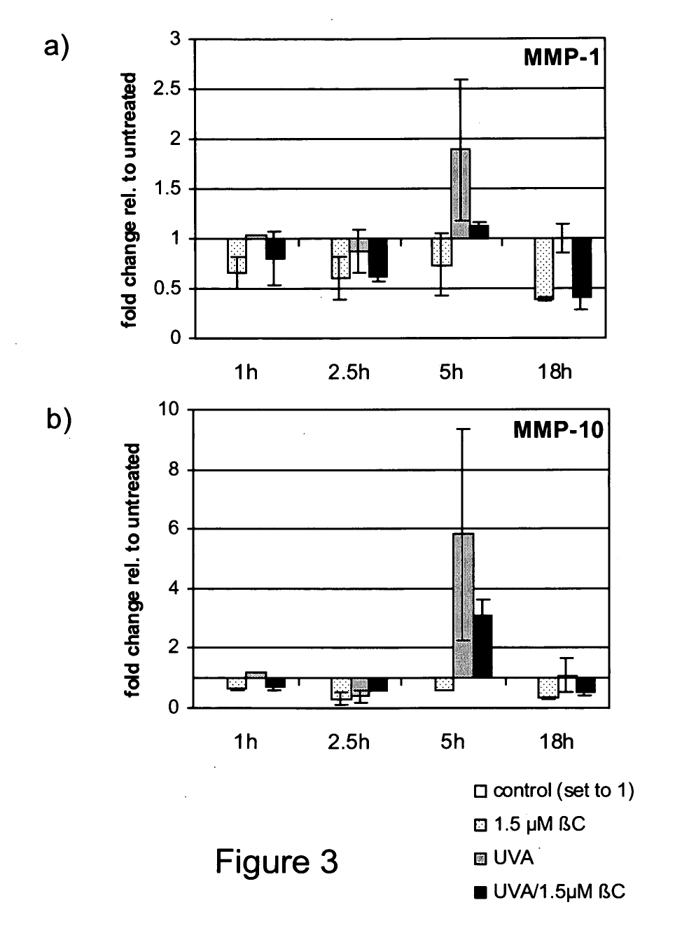

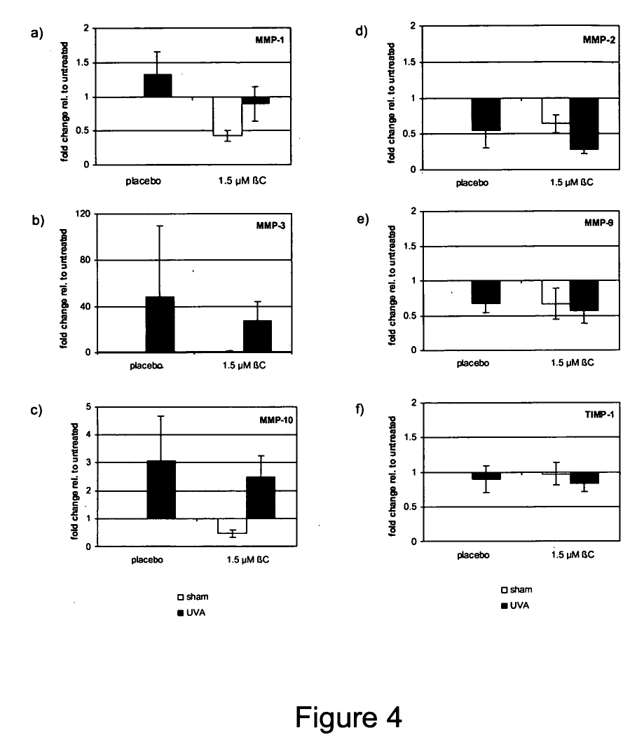

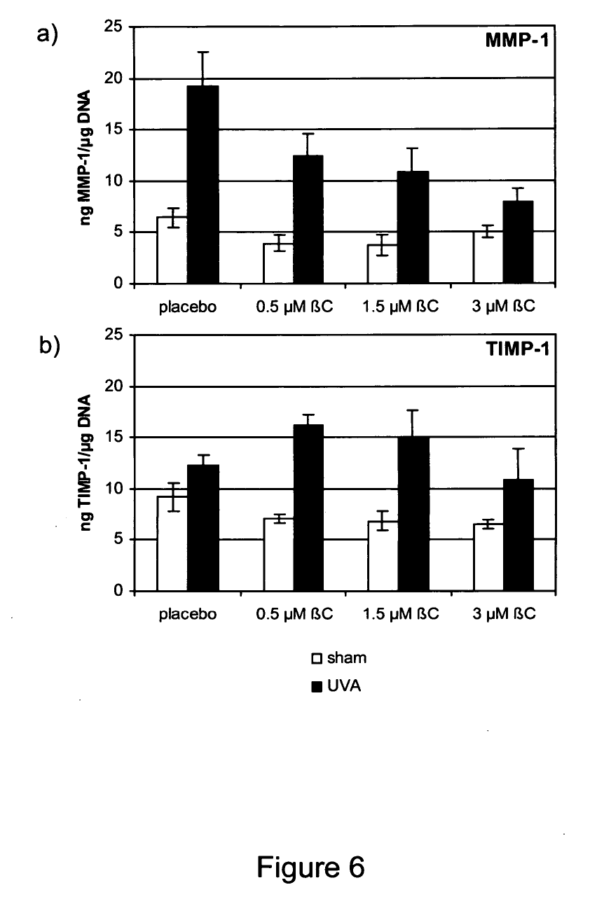

β-Carotene, like other carotenoids, is an excellent ¹O₂ quencher [66, 67]. Since ¹O₂-dependent induction of MMPs upon UVA exposure is thought to be a major mechanism of photoaging, β-carotene inhibition of MMP induction upon UVA exposure was measured. Among MMPs, MMP-1 is best characterized in terms of induction by UV light, and is the most accepted marker for photoaging. MMP-1 transcripts were present at medium to high levels in HaCaT cells, and were detected at a δCT of 12.67 in controls. We found a 2.4 fold (SE±0.7) induction of MMP-1 by UVA at 5 hours after irradiation, but only little inducibility at the other time points analyzed (FIG. 3a). Therefore, the 5 hour time point was chosen to analyze the effect of treatments on gene expression in all further experiments. The degree of UVA inducibility of MMP-1 expression varied between experiments. In any case, β-carotene at a concentration of 1.5 μM significantly reduced UVA-induced MMP-1 induction from 1.3 fold to 0.9 fold on average (FIG. 4a; P=0.047). Downregulation of UVA-induced MMP-1 production by β-carotene was also confirmed on the protein level (FIG. 6a; ANOVA P=0.005).

By microarray analysis [53] showed that among the MMP genes detected by the array, MMP-10 (stromelysin-2) was the most strongly induced by UVA in HaCaT cells at 5 hours after irradiation. Pretreatment with 1.5 μM β-carotene moderately io reduced UVA induction of MMP-10 by 30% from 4.6 fold to 3.2 fold. This result was confirmed by QRT-PCR. MMP-10 was a medium abundant transcript in untreated HaCaT cells (δCT 15.35). UVA induced MMP-10 expression to about 3 fold relative to the expression in unirradiated cells (FIG. 4c). 1.5 μM β-carotene reduced UVA induction of MMP-10 to approx. 2.5 fold, an effect that reached marginal significance (P=0.088). As with MMP-1, MMP-10 was maximally induced 5 hours after UVA irradiation (FIG. 3b; UVA effect at 5 hours P<0.0001). UVA exposure increased MMP-10 expression 5.8 fold (SE±3.56) at this time point.

MMP-3 (stromelysin-1) was analyzed as a close relative to MMP-10. MMP-3 was present at medium abundance in HaCaT cells (δCT 22.8). MMP-3 is also known to be induced by UVA1 light (340-450 nm) [68, 69]. Accordingly, MMP-3 was induced approx. 49 fold by UVA exposure, and 1.5 μM β-carotene non-significantly reduced UVA-induction of MMP-3 to 27 fold relative to unirradiated controls (FIG. 4b).

The expression profiles of the two gelatinases MMP-2 and MMP-9 were analyzed. MMP-9 is induced by UV irradiation in skin [70]. Both MMP-2 and MMP-9 were expressed at medium abundance with δCTs of 18.64 and 21.08, respectively, in controls. Unexpectedly, neither of the gelatinases was induced by this irradiation regimen, and β-carotene did not influence their expression significantly (FIGS. 4d and 4e).

TIMP-1, an endogenous MMP inhibitor, was strongly expressed (δCT 5.9), but not significantly influenced by the treatments on the RNA (FIG. 4f) or protein level (FIG. 6b).

β-Carotene Acted as a ¹O₂-Quencher in Living Cells

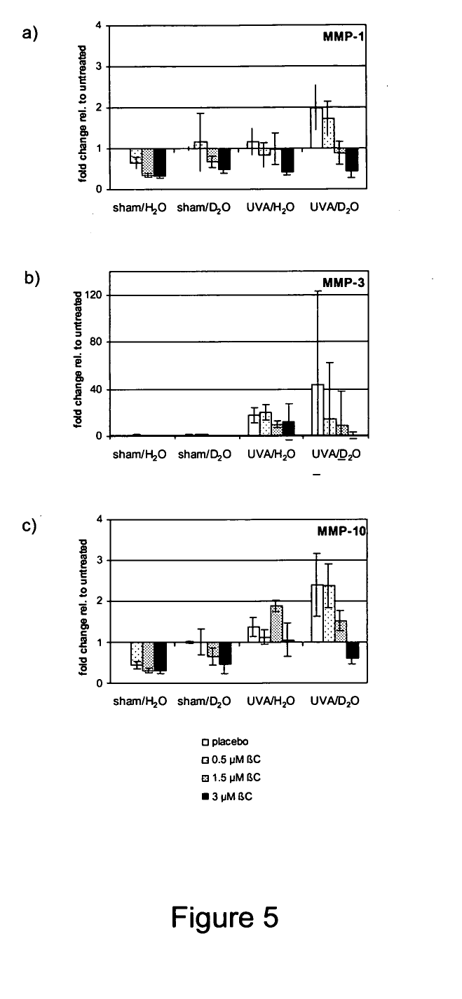

To test whether the mechanism by which β-carotene interferes with UVA induction of MMPs involves ¹O₂ quenching cells were irradiated either in D₂O-containing buffer or in H₂O-containing buffer. D₂O is able to prolong the lifetime of ¹O₂[18]. Thus, the probability of ¹O₂reacting with a relevant target is increased. Accordingly, ¹O₂-dependent MMP induction upon UVA exposure should be more pronounced after irradiation in the presence of D₂O. β-Carotene should then be able to reduce MMP induction by UVA/D₂O treatment. Wlaschek et al. [13, 15, 71] have described that MMP-1 induction by UVA involves ¹O₂-dependent mechanisms. In line with this, QRT-PCR analysis indeed revealed greater induction of MMP-1, when the cells were irradiated in the presence of D₂O (FIG. 5a; 1.9 fold vs. 1.2 fold; ANOVA P for D₂O effect=0.0505; Fisher's PLSD test D₂O vs. H₂O P=0.001 1). β-carotene significantly and dose-dependently reduced UVA/D₂O-induced MMP-1 induction (ANOVA P for β-carotene effect=0.0563; Fisher's PLSD: β-carotene at 1.5 μM P=0.0405; β-carotene at 3 μM P<0.0001). Moreover, β-carotene treatment also tended to reduce basal MMP-1 RNA and protein expression in unirradiated cells (Protein: FIG. 6a; P=0.0537).

MMP-10 is known to be induced by UV light [72]. So far, it has not been demonstrated, whether MMP-10 regulation also involves ¹O₂-dependent pathways. Provided below is evidence that MMP-10 is also a ¹O₂-regulated gene. D₂O significantly enhanced UVA induction of MMP-10 from 1.4 fold to 2.4 fold relative to unirradiated controls (FIG. 5c; ANOVA P for D₂O effect=0.0017; Fisher's PLSD H₂O vs. D₂O P=0.0004). This shows that MMP-10 induction by UVA irradiation involves ¹O₂-dependent mechanisms.

Pretreatment of cells with different doses of β-carotene opposed MMP-10 induction by UVA and D₂O in a dose-dependent fashion (ANOVA P for β-carotene effect=0.0368; Fisher's PLSD β-carotene at 3 μM P=0.0021). Like for MMP-1, β-carotene also tended to reduce the basal MMP-10 expression in unirradiated cells.

Both the expression profiles of MMP-1 and MMP-10 prove that β-carotene can act as a ¹O₂ quencher in living cells.

MMP-3 induction by UVA was enhanced by irradiation in D₂O -containing buffer from 18 fold to 43 fold (FIG. 5b). Since the degree of MMP-3 inducibility by UVA/D₂O varied between experiments, the effect of D₂O did not reach significance (ANOVA P=0.24). Although it remains unclear, whether MMP-3 regulation includes ¹O₂-dependent mechanisms, β-carotene prevented MMP-3 induction by UVA irradiation in the presence or absence of D₂O (ANOVA P=0.04; Fisher's PLSD P for β-carotene 3 μM=0.007).

MMP-2 and MMP-9 were not induced by UVA/D₂O treatment, and β-carotene had no significant effect on their expression (data not shown). For MMP-9, β-carotene tended to lower expression in irradiated and unirradiated cells (ANOVA P for β-carotene effect=0.08; Fisher's PLSD P for β-carotene 1.5 μM=0.028; P for β-carotene 3 μM=0.012).

TIMP-1 was again not significantly influenced by the treatments (data not shown).

Vitamin E did not Synergize with β-Carotene to Further Reduce MMP-1, MMP-3, or MMP-10 Expression

The chain-breaking antioxidant Vitamin E is thought to protect the ¹O₂ quencher β-carotene from destruction by other reactive oxygen species, and is therefore expected to potentiate the effect of β-carotene [73]. Thus, whether vitamin E at the physiologically relevant concentration of 50 μM synergizes with β-carotene at 1.5 μM to reduce MMP-1, MMP-3, and MMP-10 expression was tested. In at least four independent experiments, vitamin E did not cooperate with β-carotene in reducing UVA-induction of MMP-1, MMP-3, or MMP-10. In fact, vitamin E alone showed no effect on MMP-1, MMP-3, or MMP-10 expression (data not shown). TIMP-1 expression was reduced by UVA in this set of experiments (P=0.0008). UVA-suppressed TIMP-1 expression was restored by vitamin E (P=0.016; data not shown).

Weak Retinoid Activity is Generated from β-Carotene in HaCaT Cells and Reduced by UVA

By HPLC analysis, we found that HaCaT keratinocytes do not produce detectable amounts of retinol or retinyl esters from β-carotene. In contrast, apocarotenals were detected. HaCaT cells were treated with 0.5, 1.5, or 3 μM β-carotene for 2 days. Cellular contents of β-carotene and β-carotene metabolites were quantified by HPLC. The results are reported in Table 2. The cellular apocarotenal contents increased dose dependently, and amounted to maximum 5 pmol/million cells treated with 3 μM β-carotene. Moreover, a fraction of the supplemented all-E β-carotene was isomerized to (Z) isomers. The amount of (Z) isomers also increased dose-dependently, and was maximum 0.8 pmol/million cells after supplementation with 3 μM β-carotene.

TABLE 2
β-Carotene uptake and metabolism in HaCaT cells,
(pmol/10⁶ cells) (LOD—below limit of detection.)

| β-Carotene | | | | | |
|---|---|---|---|---|---|
| Supplementation | all-E-β- | (Z)-β- | | | Retinyl |
| (μM) | Carotene | Carotene | Apocarotenals | Retinol | Palmitate |
| placebo | <LOD | <LOD | <LOD | <LOD | <LOD |
| 0.5 | 9.7 ± 0.09 | 0.2 ± 0.07 | 1.18 ± 0.04 | <LOD | <LOD |
| 1.5 | 34.3 ± 0.05 | 0.41 ± 0.02 | 3.21 ± 0.19 | <LOD | <LOD |
| 3.0 | 63.90 ± 0.22 | 0.82 ± 0.16 | 5.04 ± 0.11 | <LOD | <LOD |

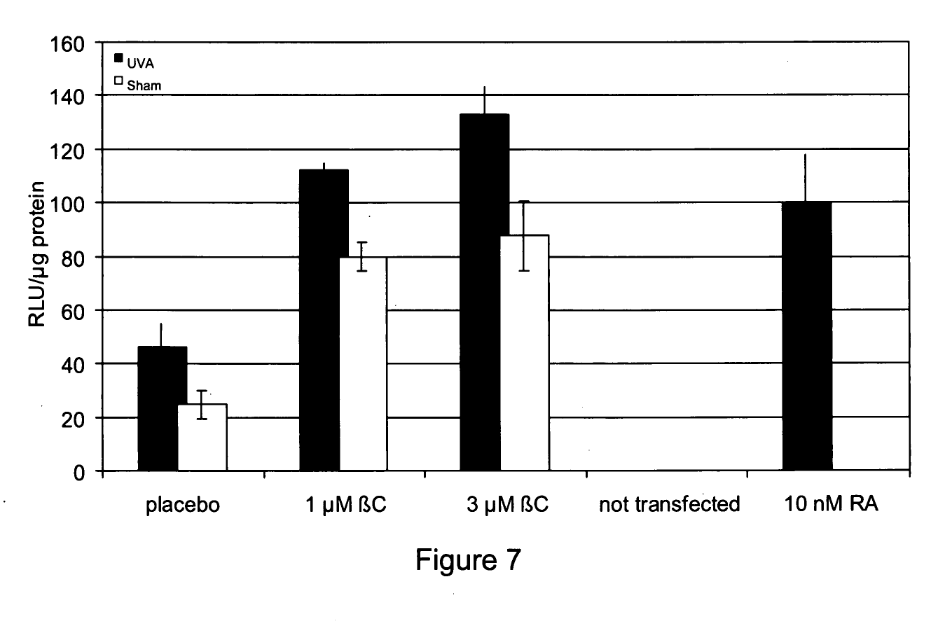

Despite undetectable retinol formation from β-carotene, RA was formed after β-carotene treatment, as shown by transactivation of an RA-dependent reporter gene (FIG. 7). Treatment of HaCaT cells with 1 or 3 μM β-carotene caused activation of the luciferase reporter to a degree comparable to what was achieved after treating the cells with a combination of all-trans RA and 9-cis RA at 10 nM each. RARE-dependent gene activation by β-carotene was reduced to about 70%, if the cells were irradiated with UVA prior to the activation measurement.

Next, results were correlated on β-carotene metabolism and RA-dependent gene activation with the expression profiles of the two β-carotene cleavage enzymes and the nuclear receptors responsible for transducing the RA effect on gene expression.

β-Carotene-15,15′-oxygenase [74-77] cleaves β-carotene centrally to yield retinal. β-Carotene-15,15′-oxygenase was expressed at a relatively low level with a δCT of 23.8 in controls. Transcripts for β-carotene-9′, 10′-oxygenase [78], which produces 10′-apocarotenal and β-ionone from β-carotene [78], were present at about 23 fold higher abundance (δCT 19.3). The RNA levels of both enzymes were not significantly influenced by the treatments.

HaCaT cells were pretreated for 2 days with 0.5, 1.5 or 3 μM β-carotene. The cells were irradiated with UVA (270 kJ/m² ) either in D₂O -containing PBS or in H₂O-containing PBS, to analyze ¹O₂ inducibility of genes. Gene expression 5 hours after UVA irradiation was analyzed by QRT-PCR. The results are reported in Table 3. Values are geometric means±standard error from three independent experiments. Upregulations greater than 1.5-fold are labelled in bold black, downregulations below 0.66-fold are bold grey.

TABLE 3
Fold induction effect of β-carotene on expression of retinoid receptors after UVA or D₂O-enhanced UVA irradiation.

| | H₂O | | | | | | | | D₂O | | | | | | | |
|---|---|---|---|---|---|---|---|---|---|---|---|---|---|---|---|---|
| | | | UVA/ | UVA/ | UVA/ | | | | | | UVA/ | UVA/ | UVA/ | | | |
| | | | βC | βC | βC | βC | βC | βC | | | βC | βC | βC | βC | βC | βC |
| Retinoid | Con- | | 0.5 | 1.5 | 3 | 0.5 | 1.5 | 3 | con- | | 0.5 | 1.5 | 3 | 0.5 | 1.5 | 3 |
| Receptor | trol | UVA | μM | μM | μM | μM | μM | μM | trol | UVA | μM | μM | μM | μM | μM | μM |
| RARα | 1.00 | 0.97 | 1.45 | 0.47 | 0.71 | 0.56 | 0.67 | 0.55 | 1.00 | 1.58 | 1.06 | 1.21 | 0.52 | 1.01 | 1.02 | 1.18 |
| RARβ | 1.00 | 0.38 | 0.44 | 0.90 | 0.86 | 0.81 | 1.20 | 1.84 | 1.00 | 1.15 | 2.49 | 1.69 | 2.35 | 1.25 | 2.46 | 2.79 |
| RARγ | 1.00 | 0.51 | 0.85 | 0.15 | 0.54 | 0.74 | 0.86 | 0.90 | 1.00 | 1.39 | 0.63 | 0.67 | 0.76 | 0.94 | 1.11 | 1.12 |
| RXRα | 1.00 | 0.57 | 0.96 | 0.27 | 0.58 | 0.89 | 0.98 | 1.13 | 1.00 | 0.79 | 0.68 | 0.31 | 0.38 | 1.25 | 0.82 | 1.06 |
| RXRβ | 1.00 | 0.63 | 0.93 | 0.71 | 1.04 | 0.67 | 0.99 | 0.99 | 1.00 | 1.32 | 1.23 | 1.09 | 0.76 | 1.32 | 1.17 | 1.61 |
| RXRγ | 1.00 | 0.45 | 0.61 | 0.08 | 0.67 | 0.39 | 0.77 | 0.33 | 1.00 | 3.17 | 1.72 | 2.70 | 1.57 | 1.47 | 2.78 | 2.57 |
| βC-15,15′- | 1.00 | 0.61 | 0.60 | 0.84 | 0.91 | 1.09 | 1.19 | 1.08 | 1.00 | 0.58 | 1.08 | 0.90 | 0.91 | 1.19 | 1.03 | 0.43 |
| oxygenase | | | | | | | | | | | | | | | | |
| βC-9′,10′- | 1.00 | 0.56 | 0.75 | 0.70 | 0.99 | 0.90 | 0.85 | 0.76 | 1.00 | 1.45 | 1.43 | 2.30 | 2.19 | 0.93 | 1.19 | 1.11 |
| oxygenase | | | | | | | | | | | | | | | | |

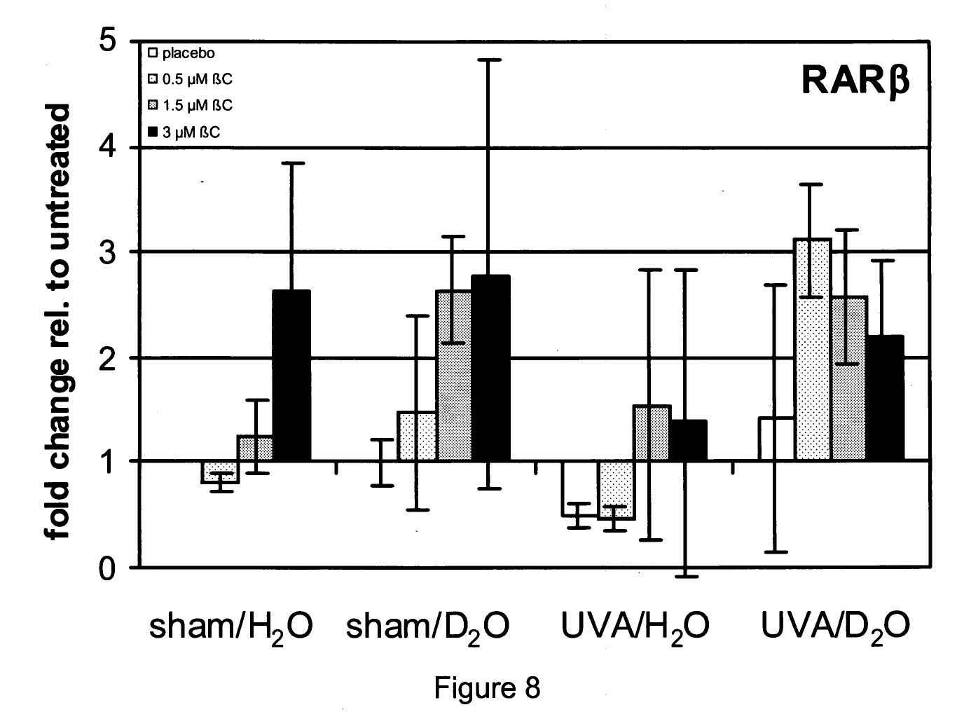

Expression of all six retinoid receptor genes (RARα, RARβ, RARγ and RXRα, RXRβ, RXRγ) was detected in HaCaT cells. RXRa was expressed the strongest among retinoid receptors with a δCT of 9.7 in controls, followed by RARα (9.8), RXRβ (13.0), RARγ (13.2), RARβ (14.5), and RXRγ (17.9). UVA downregulated all retinoid receptors approximately 2-fold, except for RARα, which was not influenced by UVA. UVA downregulation of RARs and RXRs reached significance only for RXRα. Apparently, regulation of RARα and γ expression, as well as regulation of RXRα and γ has a ¹O₂-dependent component, as D₂O treatment had a significant effect on these transcripts. β-Carotene had no significant effect on the basal or UVA-regulated expression levels of RARs and RXRs. Of note, β-carotene non-significantly induced RARβ in a dose-dependent manner, an effect observed predominantly in unirradiated cells (FIG. 8).

### CONCLUSION

The ¹O₂ quencher β-carotene alleviates UVA induction of MMP-1, MMP-3, and MMP-10, three major metalloproteases involved in premature skin aging. Moreover, the β-carotene effects were exerted mainly via RA-independent pathways. HaCaT cells produce low amounts of RA from β-carotene, as shown by monitoring RA-dependent gene activation. Thus, HaCaT cells are an excellent model to analyze the provitamin A-independent effects of β-carotene.

Time- and Dose-Dependent Accumulation of β-Carotene in HaCaT Cells

HaCaT keratinocytes took up β-carotene in a time and dose-dependent manner (FIG. 1). HaCaT cells had to be supplemented at least for two days to achieve meaningful β-carotene accumulation. The cells continued to take up β-carotene thereafter, such that maximum β-carotene levels were found after three days of supplementation. After that, daily supplementation was ceased, to monitor the cellular β-carotene content over time, if no fresh β-carotene was added. As a result, β-carotene decreased, demonstrating that a daily supply of fresh β-carotene is critical to maintain cellular β-carotene content.

UVA Irradiation Depleted Cellular β-Carotene Content

The UVA dose applied destroyed all β-carotene but about 13% of the content before irradiation, which confirms similar reports from β-carotene supplemented fibroblasts after UVA exposure [79] (FIG. 2). Consistent with this finding, RARE-dependent gene activation by β-carotene was reduced, if the cells were irradiated with UVA (FIG. 7). These results are in line with in vivo observations that UVA exposure depletes epidermal vitamin A stores [80]. Moreover, UVA irradiation reduces carotenoid concentrations in skin [24] and even in plasma [81]. In view of the role of vitamin A in maintaining skin integrity, depletion of vitamin A and provitamin A stores by UV light calls for increased vitamin A uptake in situations with extensive sun exposure.

β-Carotene Reduced Basal and ¹O₂-Induced MMP-1 and MMP-10 Induction

According to the current model of photoaging [45], UV irradiation activates growth factor and cytokine receptors, which via PKC, MAP kinases, and the NFκB pathway activate genes involved in photoaging, such as MMPs [82]. UVA1, on the other hand, is thought to induce genes associated with photoaging by ¹O₂-mediated pathways that target the transcription factor AP-2 [16]. Of the genes involved in photoaging, MMP-1 [12, 71], IL-6 [71], and ICAM-1 [16] have been shown to be induced in a ¹O₂-dependent fashion upon UVA exposure. Other reports suggest that the cellular reaction to UVA1, like UVB/UVA2, also includes activation of the stress-activated protein kinases [83-85]. Therefore, the response to UVA1 vs. UVB/UVA2 exposure, and the pathways involved, overlap. The extent to which the MMPs mainly responsible for extracellular matrix degradation are transcriptionally regulated by ¹O₂ exposure (i.e. UVA/D₂O treatment), and how the ¹O₂ quencher β-carotene would interfere with this regulation, were investigated.

That UVA induction of MMP-1 involves a ¹O₂-dependent mechanism in keratinocytes was confirmed. β-Carotene inhibited UVA/D₂O-induced MMP-1 expression in a dose-dependent manner to below control levels, demonstrating that under appropriately controlled conditions, β-carotene acts as a ¹O₂ quencher also in living cells (FIG. 5). Our results are in contrast to those reported by Obermüller-Jevic et al. [86, 87], and Offord et al. [88]. Both groups have addressed the effect of β-carotene on MMP-1 or HO-1 induction by UVA in fibroblasts. In these studies, no photoprotective effect of β-carotene was found. Rather, β-carotene enhanced UVA-induced MMP-1 and HO-1 induction. On the other hand, Trekli et aL [79] found a photoprotective effect of β-carotene against UVA irradiation in fibroblasts, as determined by HO-1 expression. These contradicting results exclude a fibroblast-specific effect, and point to experimental differences, most likely the mode of β-carotene application. In the studies, where a prooxidative effect of β-carotene was described, β-carotene was delivered to the cells either in methyl-β-cyclodextrin [86, 87], or as a nanoparticle formulation containing vitamin E [88]. Both studies, where β-carotene was photoprotective, THF containing 0.025% BHT was used as a vehicle for β-carotene [79]. A likely explanation for the different experimental outcomes is that BHT protected β-carotene better than the much lower concentration of vitamin E in the nanoparticle formulation. In the studies by Obermüller et al., β-carotene was added to the cells without antioxidant protection. In addition, the vehicle methyl-β-cyclodextrin used by Obermüller et al. is known to remove cholesterol from the cell membranes [89, 90], with drastic consequences for cell signaling events. Although it appears that the major difference is the use of BHT-containing solvent for β-carotene, the presence of the photoprotective effect of β-carotene in the present studies was not due to the protection of β-carotene by BHT. But rather that replacement with fresh β-carotene-containing medium each day and after irradiation was crucial to remove β-carotene degradation products.

Further support for a photoprotective effect of β-carotene comes from the finding that β-carotene protects against mitochondrial common deletions, a mitochondrial DNA mutation, which is induced by repeated UVA irradiation and is associated with photoaging [91]. Protection of fibroblasts against UVB irradiation by β-carotene was demonstrated by Eichler et aL. [92].

MMP-10 is a ¹O₂-induced gene (FIG. 5c). As with MMP-1, β-carotene dose-dependently inhibited UVA/D₂O -induced MMP-10 induction. For both MMP-1 and MMP-10, β-carotene also tended to reduce expression in unirradiated cells, pointing towards a preventive role of β-carotene against intrinsic skin aging. MMP-10 was more strongly induced by UVA than was MMP-1. However, the overall expression profiles of MMP-1 and MMP-10 were remarkably similar, indicating co-regulation. This is in contrast to the RNA expression profiles of the two gelatinases MMP-2 and MMP-9, which were not induced by the irradiation regimen, and which were also not regulated by β-carotene.

The stromelysin MMP-3, which is highly related to MMP-10, was strongly induced by UVA and UVA/D₂O. β-Carotene had a significant reducing effect on MMP-3 expression, although the D₂O effect on MMP-3 induction did not reach significance. The expression profile indicates that MMP-3 regulation may involve ¹O₂-dependent pathways, and Herrmann et al. also suggested that MMP-3 is ¹O₂-inducible [69]. Other mechanisms appear to dominate, however. It is unclear, whether β-carotene reduction of UVA and UVA/D₂O -induced MMP-3 expression was due to its ¹O₂ quenching ability or whether other mechanisms were involved.

Of the three MMPs regulated by UVA and β-carotene, MMP-1 was by far the strongest expressed. MMP-1 mRNA levels were approximately 6 fold higher than those of MMP-10, and 1000 fold higher than those of MMP-3. MMP-1 has a dominant role in UVA-induced degradation of fibrillar collagen, especially of collagen types III and I [93, 94]. Brennan et al. [95] found that blocking antibodies to MMP-1 removed 95% of the collagenolytic activity in the organ culture fluid from UV-treated skin. MMP3 and MMP-10 have broader substrate specificity than MMP-1, and cleave collagen IV, fibronectin, aggrecan and nidogen. Most importantly, both MMP-3 and MMP-10 have an additional role in activating other MMPs, including MMP-1 [93]. Thus, despite their lower expression level in comparison with MMP-1, they have a major impact on ECM degradation. The combined reduction by β-carotene of UVA-induced expression of MMP1, 3, and 10 indicates that β-carotene has a physiologically relevant photoprotective effect.

Vitamin E did not Synergize with β-Carotene to Further Reduce MMP-1, MMP-3, or MMP-10 Expression

The absence of a synergistic effect of vitamin E and β-carotene may be explained by sufficient amounts of intact β-carotene being present for protection against ¹O₂-mediated MMP induction under our culture conditions, even if some β-carotene was destroyed by oxidative breakdown. The finding that vitamin E alone did not reduce UVA/D₂O -induced expression of any of the MMPs tested is less easily explained, since it has been shown that vitamin E also inhibited some UVA (¹O₂)-induced mechanisms, such as common mitochondrial deletions [96]. Although we did not measure the cellular vitamin E content, it has been shown that HaCaT cells are able to accumulate vitamin E [97], arguing that our findings are not due to a lack of vitamin E uptake. Like β-carotene, vitamin E was reported to be destroyed by UV light in skin [98, 99].

Weak Retinoid Activity is Generated from β-Carotene in HaCaT Cells and Reduced by UVA

β-Carotene served as a precursor for RA in HaCaT cells, although only to a minor degree, as demonstrated by the transactivation of an RA-dependent reporter gene. No retinol or retinyl esters were detected after β-carotene supplementation in HaCaT cells. This is consistent with the low expression level of the central β-carotene cleavage enzyme, β-carotene-15,15′-oxygenase. In addition, Torma et al. have shown defective retinol esterification in HaCaT cells [100]. Also, HaCaT cells are known to express the RA-degrading enzyme CYP26 at high levels [101]. Such a constellation should cause the low amounts of RA formed from β-carotene to be rapidly degraded, leaving trace amounts of RA for gene regulation. Eccentric cleavage products of β-carotene, apocarotenals, were present at detectable concentrations in HaCaT cells. Although apocarotenals can also be formed by oxidative breakdown, their prevalence is in accord with the higher expression of the eccentric cleavage enzyme β-carotene-9′,10′-oxygenase. Apocarotenals can be metabolized to RA via β-oxidation [102], and may well serve as the precursors for the RA that was indirectly detected by monitoring gene regulation. There is only scarce information available for the regulation of the two cloned β-carotene cleavage enzymes, β-carotene-15,15′-oxygenase [103, 104] and β-carotene-9′,10′-oxygenase. Both enzymes were not influenced by the treatments on the RNA level. β-Carotene-15,15′-oxygenase activity in duodenum, a tissue with high β-carotene cleaving activity, is suppressed by β-carotene, apo-8′-carotenal, retinol, or RA in rats [103]. Takeda et al. reported that β-carotene-15,15′-oxygenase activity was induced in skin of UV-irradiated SKH-1 hairless mice [105]. In HaCaT cells, this regulation is less obvious, most likely due to marginal RA production from β-carotene in HaCaT cells.

To differentiate the pro-retinoid effects of β-carotene in interaction with UVA from its ¹O₂ quenching, the gene expression profiles of RARs and RXRs, which are required to transduce the RA effects, were characterized. Moreover, RARβ represents one of the best characterized RA target genes. Human epidermis expresses RARα, RARγ, RXRα, and RXRβ, as detected by Northern blot analysis [106]. Transcript levels for RARβ reportedly are low or undetectable, and RXRγ RNA was not detected. HaCaT cells were shown to express RARβ, in addition to RARα, RARγ, and RXRα [100]. Thus, HaCaT cells express all six retinoid receptor genes.

UVA downregulation of retinoid receptors is in line with reports from Wang et al. [107]. They showed that UV irradiation of human skin causes downregulation of RARγ and RXRα, which can be prevented by pretreatment with RA. β-Carotene had no significant effect on the expression levels of RARγ and RXRα, but non-significantly induced RARβ in a dose-dependent manner. This result is consistent with low amounts of RA being formed from β-carotene, which suffice for a mild induction of the RA target gene RARβ [108, 109], and for induction of the extremely sensitive artificial promoter of the reporter gene containing five RAREs. The RARs and RXRs other than RARβ also contain autoregulatory elements in their promoters [110-112], but they are much less sensitive to induction by RA than RARβ.

In addition to activating RARE-dependent transcription, RA inhibits gene expression by transrepression of AP-1. Since MMP induction by UV light is mainly regulated by AP-2 and AP-1, RA would be expected to suppress UV-induced MMP expression. Indeed, Fisher and Voorhees have shown that UV(B) induction of MMPs 1, 3, and 9 in human skin can be prevented by RA pretreatment [70]. At the same time, DNA binding by AP-1 was reduced. The dose response curve for AP-1 transrepression by RA is not necessarily identical to transactivation of an RARE, or of a reporter construct driven by 5 RAREs. For two fibroblast cell lines collagenase expression is reduced by 10 nM RA [113, 114]. However, 1, 10, or 100 nM RA had no effect on UVA-induced MMP-1 secretion in this system (Goralczyk, unpublished observations). This rules out that downregulation of UVA-induced MMP-1 expression is mediated by β-carotene-derived retinoid activity.

Moreover, RA, and 1,25-dihydroxyvitamin D3 (1,25-(OH)₂D3), may play a role in the regulation of the genes analyzed in this study. HaCaT cells were shown to synthesize 1,25-(OH)₂D3 upon stimulation, e.g., with EGF [115]. Since the cellular response to UV involves an activation of the EGFR and downstream signalling pathways, UVA irradiation may well increase 1,25-(OH)₂D3 synthesis in HaCaT cells.

In rheumatoid synovial fibroblasts, 1,25-(OH)₂D3 inhibited IL1-induced MMP-1 and MMP-3 secretion [116]. If this is also the case in HaCaT cells, it would imply that MMP-1 and 3 induction by UVA would be even higher than if no 1,25-(OH)₂D3 was synthesized upon UVA irradiation. If (all-trans)-β-carotene contributes to increased 9-cis RA formation, such an increased ligand concentration of both 1,25-(OH)₂D3 and 9-cis RA could mediate a further decrease of MMP expression and thus contribute to the photoprotective effect of β-carotene. On the other hand, the microarray data show that UVA irradiation caused a downregulation of both VDR and RXRA (both downregulated by about 50%; [53]), indicating that the VDR system does not play a major role in this setting.

In sum, β-carotene suppressed UVA-induction of MMP-1, MMP-3, and MMP-10, which represent matrix metalloproteases crucially involved in degradation of the extracellular matrix during premature skin aging. Not only MMP-1, but also MMP-10 is regulated by ¹O₂-dependent pathways, and that β-carotene quenched ¹O₂-mediated induction of both MMP-1 and MMP-10. Vitamin E did not cooperate with β-carotene to further reduce UVA-induced MMP-1, MMP-3, or MMP-10 expression. HaCaT cells produced minute amounts of compounds with retinoid activity from β-carotene, as detected by marginal induction of RARβ and an RARE-dependent reporter gene. This feature renders HaCaT cells an excellent cell system to dissect and characterize the effect of the intact β-carotene molecule from the vitamin A activity of its metabolites.

### BIBLIOGRAPHY

1. Berneburg, M.; Clingen, P. H.; Harcourt, S. A.; Lowe, J. E.; Taylor, E. M.; Green, M. H.; Krutmann, J.; Arlett, C. F. and Lehmann, A. R. The cancer-free phenotype in trichothiodystrophy is unrelated to its repair defect. Cancer Res 60:431-8.; 2000.
2. Kligman, A. M. Early destructive effect of sunlight on human skin. JAMA 210:2377-80; 1969.
3. IARC, eds. Solar and UV radiation. Lyon: IARC; 1992
4. Freeman, R. G. Data on the action spectrum for ultraviolet carcinogenesis. J Natl Cancer Inst 55:11 1,9-22; 1975.
5. Tyrrell, R. M. Activation of mammalian gene expression by the UV component of sunlight—from models to reality. Bioessays 18:139-48; 1996.
6. Tyrrell, R. M. UV activation of mammalian stress proteins. Exs 77:255-71; 1996.
7. Yasui, H. and Sakurai, H. Chemiluminescent detection and imaging of reactive oxygen species in live mouse skin exposed to UVA. Biochem Biophys Res Commun 269:131-6; 2000.
8. Black, H. S. Potential involvement of free radical reactions in ultraviolet light-mediated cutaneous damage. Photochem Photobiol 46:213-21; 1987.
9. Dalle Carbonare, M. and Pathak, M. A. Skin photosensitizing agents and the role of reactive oxygen species in photoaging. J Photochem Photobiol B 14:105-24; 1992.
10. Grether-Beck, S. and Krutmann, J. Gene regulation by ultraviolet A radiation and singlet oxygen. Methods Enzymol 319:280-90; 2000.
11. Basu-Modak, S. and Tyrrell, R. M. Singlet oxygen: a primary effector in the ultraviolet A/near-visible light induction of the human heme oxygenase gene. Cancer Res 53:4505-10; 1993.
12. Scharffetter-Kochanek, K.; Wlaschek, M.; Briviba, K. and Sies, H. Singlet oxygen induces collagenase expression in human skin fibroblasts. FEBS Lett 331:304-6; 1993.
13. Wlaschek, M.; Briviba, K.; Stricklin, G. P.; Sies, H. and Scharffetter-Kochanek, K. Singlet oxygen may mediate the ultraviolet A-induced synthesis of interstitial collagenase. J Invest Dermatol 104:194-8; 1995.
14. Keyse, S. M. and Tyrrell, R. M. Heme oxygenase is the major 32-kDa stress protein induced in human skin fibroblasts by UVA radiation, hydrogen peroxide, and sodium arsenite. Proc Natl Acad Sci USA 86:99-103; 1989.
15. Wlaschek, M.; Heinen, G.; Poswig, A.; Schwarz, A.; Krieg, T. and Scharffetter-Kochanek, K. UVA-induced autocrine stimulation of fibroblast-derived collagenase/MMP-1 by interrelated loops of interleukin-1 and interleukin-6. Photochem Photobiol 59:550-6; 1994.
16. Grether-Beck, S.; Olaizola-Horn, S.; Schmitt, H.; Grewe, M.; Jahnke, A.; Johnson, J. P.; Briviba, K.; Sies, H. and Krutmann, J. Activation of transcription factor AP-2 mediates UVA radiation- and singlet oxygen-induced expression of the human intercellular adhesion molecule 1 gene. Proc Natl Acad Sci USA 93:14586-91; 1996.
17. Foote, C. S. Mechanisms of photosensitized oxidation. There are several different types of photosensitized oxidation which may be important in biological systems. Science 162:963-970; 1968.
18. Merkel, P.; Nilsson, R. and Kearns, D. R. Deuterium effects on singlet oxygen lifetime in solutions. A new test of singlet oxygen reactions. J Am Chem Soc 94:1030-1031; 1972.
19. Truscott, T. The photophysics and photochemistry of the carotenoids. J Photochem Photobiol B:Biol 6:359-371; 1990.
20. Conn, P. F.; Lambert, C.; Land, E. J.; Schalch, W. and Truscott, T. G. Carotene-oxygen radical interactions. Free Radic Res Commun 16:401-8; 1992.
21. Conn, P. F.; Schalch, W. and Truscoff, T. G. The singlet oxygen and carotenoid interaction. J Photochem Photobiol B 11:41-7; 1991.
22. Vahlquist, A.; Lee, J. B.; Michaelsson, G. and Rollman, O. Vitamin A in human skin: II Concentrations of carotene, retinol and dehydroretinol in various components of normal skin. J Invest Dermatol 79:94-7; 1982.
23. Lee, R.; Mathews-Roth, M. M.; Pathak, M. A. and Parrish, J. A. The detection of carotenoid pigments in human skin. J Invest Dermatol 64:175-7; 1975.
24. Biesalski, H. K.; Hemmes, C.; Hopfenmuller, W.; Schmid, C. and Gollnick, H. P. Effects of controlled exposure of sunlight on plasma and skin levels of beta-carotene. Free Radic Res 24:215-24; 1996.
25. Davison, P. A.; Hunter, C. N. and Horton, P. Overexpression of beta-carotene hydroxylase enhances stress tolerance in Arabidopsis. Nature 418:203-6; 2002.
26. Mathews-Roth, M. M.; Pathak, M. A.; Parrish, J.; Fitzpatrick, T. B.; Kass, E. H.; Toda, K. and Clemens, W. A clinical trial of the effects of oral beta-carotene on the responses of human skin to solar radiation. J Invest Dermatol 59:349-53; 1972.
27. Mathews-Roth, M. M.; Pathak, M. A.; Fitzpatrick, T. B.; Harber, L. H. and Kass, E. H. Beta carotene therapy for erythropoietic protoporphyria and other photosensitivity diseases. Arch Dermatol 113:1229-32; 1977.
28. Mathews-Roth, M. M. Erythropoietic protoporphyria: treatment with antioxidants and potential cure with gene therapy. Methods Enzymol 319:479-84; 2000.
29. Anstey, A. V.; Ryan, A.; Rhodes, L. E.; Charman, C. R.; Arlett, C. F.; Tyrrell, R. M.; Taylor, C. R. and Pearse, A. D. Characterization of photosensitivity in the Smith-Lemli-Opitz syndrome: a new congenital photosensitivity syndrome. Br J Dermatol 141:406-14; 1999.
30. Thomas, J. P.; Hall, R. D. and Girotti, A. W. Singlet oxygen intermediacy in the photodynamic action of membrane-bound hematoporphyrin derivative. Cancer Lett 35:295-302; 1987.
31. Heinrich, U.; Gartner, C.; Wiebusch, M.; Eichler, O.; Sies, H.; Tronnier, H. and Stahl, W. Supplementation with beta-Carotene or a Similar Amount of Mixed Carotenoids Protects Humans from UV-lnduced Erythema. J Nutr 133:98-101; 2003.
32. Stahl, W. and Sies, H. Carotenoids and protection against solar UV radiation. Skin Pharmacol Appl Skin Physiol 15:291-6; 2002.
33. Stahl, W.; Heinrich, U.; Jungmann, H.; Sies, H. and Tronnier, H. Carotenoids and carotenoids plus vitamin E protect against ultraviolet light-induced erythema in humans. Am J Clin Nutr 71:795-8; 2000.
34. Alaluf, S.; Heinrich, U.; Stahl, W.; Tronnier, H. and Wiseman, S. Dietary carotenoids contribute to normal human skin color and UV photosensitivity. J Nutr 132:399-403; 2002.
35. Gollnick, H. P. M.; Hopfenmuiller, W.; Hemmes, C.; Chun, S. C.; Schmid, C.; Sundermeier, K. and Biesalski, H. K. Systemic β-carotene plus topical UV-sunscreen are an optimal protection against harmful effects of natural UV-sunlight: results of the Berlin-Eilath study. Eur J Dermatol 6:200-205; 1996.
36. Lee, J.; Jiang, S.; Levine, N. and Watson, R. R. Carotenoid supplementation reduces erythema in human skin after simulated solar radiation exposure. Proc Soc Exp Biol Med 223:170-4; 2000.
37. Bialy, T. L.; Rothe, M. J. and Grant-Kels, J. M. Dietary factors in the prevention and treatment of nonmelanoma skin cancer and melanoma. Dermatol Surg 28:1143-52; 2002.
38. Sayre, R. M. and Black, H. S. Beta-carotene does not act as an optical filter in skin. J Photochem Photobiol B 12:83-90; 1992.
39. Wei, R. R.; Wamer, W. G.; Lambert, L. A. and Kornhauser, A. beta-Carotene uptake and effects on intracellular levels of retinol in vitro. Nutr Cancer 30:53-8; 1998.
40. Andersson, E.; Vahlquist, A. and Rosdahl, I. Beta-carotene uptake and bioconversion to retinol differ between human melanocytes and keratinocytes. Nutr Cancer 39:300-6; 2001.
41. Griffiths, C. E.; Kang, S.; Ellis, C. N.; Kim, K. J.; Finkel, L. J.; Ortiz-Ferrer, L. C.; White, G. M.; Hamilton, T. A. and Voorhees, J. J. Two concentrations of topical tretinoin (retinoic acid) cause similar improvement of photoaging but different degrees of irritation. A double-blind, vehicle-controlled comparison of 0.1% and 0.025% tretinoin creams. Arch Dermatol 131:1037-44; 1995.
42. Kligman, A. M. Topical retinoic acid (tretinoin) for photoaging: conceptions and misperceptions. Cutis 57:1424; 1996.
43. Fisher, G. J.; Wang, Z. Q.; Datta, S. C.; Varani, J.; Kang, S. and Voorhees, J. J. Pathophysiology of premature skin aging induced by ultraviolet light. N Engl J Med 337:1419-28; 1997.
44. Fisher, G. J.; Talwar, H. S.; Lin, J.; Lin, P.; McPhillips, F.; Wang, Z.; Li, X.; Wan, Y.; Kang, S. and Voorhees, J. J. Retinoic acid inhibits induction of c-Jun protein by ultraviolet radiation that occurs subsequent to activation of mitogen-activated protein kinase pathways in human skin in vivo. J Clin Invest 101:1432-40; 1998.
45. Fisher, G. J.; Talwar, H. S.; Lin, J. and Voorhees, J. J. Molecular mechanisms of photoaging in human skin in vivo and their prevention by all-trans retinoic acid. Photochem Photobiol 69:154-7; 1999.
46. Fisher, G. J.; Datta, S.; Wang, Z.; Li, X. Y.; Quan, T.; Chung, J. H.; Kang, S. and Voorhees, J. J. c-Jun-dependent inhibition of cutaneous procollagen transcription following ultraviolet irradiation is reversed by all-trans retinoic acid. J Clin Invest 106:663-70; 2000.
47. Griffiths, C. E. Dowling Oration delivered at the Royal College of Physicians, London, Friday 5 Jun. 1998. Retinoids: renaissance and reformation. Clin Exp Dermatol 24:329-35; 1999.
48. Kang, S.; Duell, E. A.; Fisher, G. J.; Dafta, S. C.; Wang, Z. Q.; Reddy, A. P.; Tavakkol, A.; Yi, J. Y.; Griffiths, C. E.; Elder, J. T. and et al. Application of retinol to human skin in vivo induces epidermal hyperplasia and cellular retinoid binding proteins characteristic of retinoic acid but without measurable retinoic acid levels or irritation. J Invest Dermatol 105:549-56; 1995.
49. Fuchs, E. Epidermal differentiation and keratin gene expression. J Cell Sci Suppl 17:197-208; 1993.
50. Breitkreutz, D.; Stark, H. J.; Plein, P.; Baur, M. and Fusenig, N. E. Differential modulation of epidermal keratinization in immortalized (HaCaT) and tumorigenic human skin keratinocytes (HaCaT-ras) by retinoic acid and extracellular Ca2+. Differentiation 54:201-17; 1993.
51. Varani, J.; Zeigler, M.; Dame, M. K.; Kang, S.; Fisher, G. J.; Voorhees, J. J.; Stoll, S. W. and Elder, J. T. Heparin-binding epidermal-growth-factor-like growth factor activation of keratinocyte ErbB receptors Mediates epidermal hyperplasia, a prominent side-effect of retinoid therapy. J Invest Dermatol 117:1335-41; 2001.
52. Frederick, J. and Alberts, A. The natural UVA radiation environment. In: Urbach, F., eds. Biological responses to UV A radiation. Kansas: Valdenmar Publishing; 1992:7-18
53. Wertz, K.; Buchwald Hunziker, P.; Seifert, N.; Riss, G.; Neeb, M.; Steiner, G.; Hunziker, W. and Goralczyk, R. β-Carotene interferes with UVA-induced gene expression by multiple pathways. in prep. 2003.
54. Ahn, K. S.; Moon, K. Y.; Lee, J. and Kim, Y. S. Downregulation of NF-kappaB activation in human keratinocytes by melanogenic inhibitors. J Dermatol Sci 31:193-201; 2003.
55. Fukunaga, M.; Oka, M.; Ichihashi, M.; Yamamoto, T.; Matsuzaki, H. and Kikkawa, U. UV-induced tyrosine phosphorylation of PKC delta and promotion of apoptosis in the HaCaT cell line. Biochem Biophys Res Commun 289:573-9; 2001.
56. Chen, W.; Tang, Q.; Gonzales, M. S. and Bowden, G. T. Role of p38 MAP kinases and ERK in mediating ultraviolet-B induced cyclooxygenase-2 gene expression in human keratinocytes. Oncogene 20:3921-6; 2001.
57. Saliou, C.; Kitazawa, M.; McLaughlin, L.; Yang, J. P.; Lodge, J. K.; Tetsuka, T.; lwasaki, K.; Cillard, J.; Okamoto, T. and Packer, L. Antioxidants modulate acute solar ultraviolet radiation-induced NF-kappa-B activation in a human keratinocyte cell line. Free Radic Biol Med 26:174-83; 1999.
58. Assefa, Z.; Garmyn, M.; Bouillon, R.; Merlevede, W.; Vandenheede, J. R. and Agostinis, P. Differential stimulation of ERK and JNK activitiesby ultraviolet B irradiation 10 and epidermal growth factor in human keratinocytes. J Invest Dermatol 108:886-91; 1997.
59. Sitailo, L. A.; Tibudan, S. S. and Denning, M. F. Activation of caspase-9 is required for UV-induced apoptosis of human keratinocytes. J Biol Chem 277:19346-52; 2002.
60. Leccia, M. T.; Richard, M. J.; Joanny-Crisci, F. and Beani, J. C. UV-A1 cytotoxicity and antioxidant defence in keratinocytes and fibroblasts. Eur J Dermatol 8:478-82; 1998.
61. Li, D.; Turi, T. G.; Schuck, A.; Freedberg, l. M.; Khitrov, G. and Blumenberg, M. Rays and arrays: the transcriptional program in the response of human epidermal keratinocytes to UVB illumination. Faseb J 15:2533-5; 2001.
62. Takao, J.; Ariizumi, K.; Dougherty, II and Cruz, P. D., Jr. Genomic scale analysis of the human keratinocyte response to broad-band ultraviolet-B irradiation. Photodermatol Photoimmunol Photomed 18:5-13; 2002.
63. Djavaheri-Mergny, M.; Mergny, J. L.; Bertrand, F.; Santus, R.; Maziere, C.; Dubertret, L. and Maziere, J. C. Ultraviolet-A induces activation of AP-1 in cultured human keratinocytes. FEBS Lett 384:92-6; 1996.
64. Boukamp, P.; Petrussevska, R. T.; Breitkreutz, D.; Hornung, J.; Markham, A. and Fusenig, N. E. Normal keratinization in a spontaneously immortalized aneuploid human keratinocyte cell line. J Cell Biol 106:761-71; 1988.
65. Edstrom, D. W.; Porwit, A. and Ros, A. M. Effects on human skin of repetitive ultraviolet-A1 (UVA1) irradiation and visible light. Photodermatol Photoimmunol Photomed 17:66-70; 2001.
66. Cantrell, A.; McGarvey, D. J.; George Truscott, T.; Rancan, F. and Bohm, F. Singlet oxygen quenching by dietary carotenoids in a model membrane environment. Arch Biochem Biophys 412:47-54; 2003.
67. Truscott, G. Reduction potential and radical reactions of carotenoids. Meeting abstract. 13th International Carotenoid Symposium, Honolulu, 2002.
68. Yin, L.; Morita, A. and Tsuji, T. Alterations of extracellular matrix induced by tobacco smoke extract. Arch Dermatol Res 292:188-94; 2000.
69. Herrmann, G.; Wlaschek, M.; Bolsen, K.; Prenzel, K.; Goerz, G. and Scharffefter-Kochanek, K. Photosensitization of uroporphyrin augments the ultraviolet A-induced synthesis of matrix metalloproteinases in human dermal fibroblasts. J Invest Dermatol 107:398-403; 1996.
70. Fisher, G. J.; Datta, S. C.; Talwar, H. S.; Wang, Z. Q.; Varani, J.; Kang, S. and Voorhees, J. J. Molecular basis of sun-induced premature skin ageing and retinoid antagonism. Nature 379:335-9; 1996.
71. Wlaschek, M.; Wenk, J.; Brenneisen, P.; Briviba, K.; Schwarz, A.; Sies, H. and Scharffetter-Kochanek, K. Singlet oxygen is an early intermediate in cytokine-dependent ultraviolet-A induction of interstitial collagenase in human dermal fibroblasts in vitro. FEBS Lett 413:239-42; 1997.
72. Gack, S.; Vallon, R.; Schaper, J.; Ruther, U. and Angel, P. Phenotypic alterations in fos-transgenic mice correlate with changes in Fos/Jun-dependent collagenase type I expression. Regulation of mouse metalloproteinases by carcinogens, tumor promoters, cAMP, and Fos oncoprotein. J Biol Chem 269:10363-9; 1994.
73. Edge, R.; Land, E. J.; McGarvey, D.; Mulroy, L. and Truscott, T. G. Relative one-electron reduction potentials of carotenoid radical cations with the vitamin E radical cation. J Am Chem Soc 120:1998.
74. Paik, J.; During, A.; Harrison, E. H.; Mendelsohn, C. L.; Lai, K. and Blaner, W. S. Expression and characterization of a murine enzyme able to cleave beta-carotene. The formation of retinoids. J Biol Chem 276:32160-8; 2001.
75. Yan, W.; Jang, G. F.; Haeseleer, F.; Esumi, N.; Chang, J.; Kerrigan, M.; Campochiaro, M.; Campochiaro, P.; Palczewski, K. and Zack, D. J. Cloning and characterization of a human beta,beta-carotene-15,15′-dioxygenase that is highly expressed in the retinal pigment epithelium. Genomics 72:193-202; 2001.
76. von Lintig, J. and Wyss, A. Molecular analysis of vitamin A formation: cloning and characterization of beta-carotene 15,15′-dioxygenases. Arch Biochem Biophys 385:47-52; 2001.
77. Wyss, A.; Wirtz, G.; Woggon, W.; Brugger, R.; Wyss, M.; Friedlein, A.; Bachmann, H. and Hunziker, W. Cloning and expression of beta, beta-carotene 15,15′-dioxygenase. Biochem Biophys Res Commun 271:334-6; 2000.
78. Kiefer, C.; Hessel, S.; Lampert, J. M.; Vogt, K.; Lederer, M. O.; Breithaupt, D. E. and von Lintig, J. Identification and characterization of a mammalian enzyme catalyzing the asymmetric oxidative cleavage of provitamin A. J Biol Chem 276:14110-6; 2001.
79. Trekli, M. C.; Riss, G.; Goralczyk, R. and Tyrrell, R. M. Beta-carotene suppresses UVA-induced HO-1 gene expression in cultured FEK4. Free Radic Biol Med 34:456-464; 2003.
80. Sorg, O.; Tran, C.; Carraux, P.; Didierjean, L.; Falson, F. and Saurat, J. H. Oxidative stress-independent depletion of epidermal vitamin A by UVA. J Invest Dermatol 118:513-8; 2002.
81. Roe, D. A. Photodegradation of carotenoids in human subjects. Fed Proc 46:1886-9; 1987.
82. Brenneisen, P.; Sies, H. and Scharffetter-Kochanek, K. Ultraviolet-B irradiation and matrix metalloproteinases: from induction via signaling to initial events. Ann N Y Acad Sci973:31-43; 2002.
83. Kick, G.; Messer, G.; Plewig, G.; Kind, P. and Goetz, A. E. Strong and prolonged induction of c-jun and c-fos proto-oncogenes by photodynamic therapy. Br J Cancer 74:30-6; 1996.
84. Gange, R. W. Comparison of pigment responses in human skin to UVB and UVA radiation. Prog Clin Biol Res 256:475-85; 1988.
85. Ariizumi, K.; Bergstresser, P. R. and Takashima, A. Wavelength-specific induction of immediate early genes by ultraviolet radiation. J Dermatol Sci 12:147-55; 1996.
86. Obermuller-Jevic, U. C.; Francz, P. I.; Frank, J.; Flaccus, A. and Biesalski, H. K. Enhancement of the UVA induction of haem oxygenase-1 expression by beta-carotene in human skin fibroblasts. FEBS Lett 460:212-6; 1999.
87. Obermuller-Jevic, U. C.; Schlegel, B.; Flaccus, A. and Biesalski, H. K. The effect of beta-carotene on the expression of interleukin-6 and heme oxygenase-1 in UV-irradiated human skin fibroblasts in vitro. FEBS Lett 509:186-90; 2001.
88. Offord, E. A.; Gautier, J. C.; Avanti, O.; Scaletta, C.; Runge, F.; Kramer, K. and Applegate, L. A. Photoprotective potential of lycopene, beta-carotene, vitamin E, vitamin C and carnosic acid in UVA-irradiated human skin fibroblasts. Free Radic Biol Med 32:1293-303; 2002.
89. Moran, M. and Miceli, M. C. Engagement of GPI-linked CD48 contributes to TCR signals and cytoskeletal reorganization: a role for lipid rafts in T cell activation. Immunity 9:787-96; 1998.
90. Roy, S.; Luetterforst, R.; Harding, A.; Apolloni, A.; Etheridge, M.; Stang, E.; Rolls, B.; Hancock, J. F. and Parton, R. G. Dominant-negative caveolin inhibits H-Ras function by disrupting cholesterol-rich plasma membrane domains. Nat Cell Biol 1:98-105; 1999.
91. Eicker, J.; Kurten, V.; Wild, S.; Riss, G.; Goralczyk, R.; Krutmann, J. and Berneburg, M. Betacarotene supplementation protects from photoaging-associated mitochondrial DNA mutation. Photochem Photobiol Sci 2:655-9; 2003.
92. Eichler, O.; Sies, H. and Stahl, W. Divergent optimum levels of lycopene, beta-carotene and lutein protecting against UVB irradiation in human fibroblastst. Photochem Photobiol 75:503-6; 2002.
93. Kahari, V. M. and Saarialho-Kere, U. Matrix metalloproteinases in skin. Exp Dermatol 6:199-213; 1997.
94. Sternlicht, M. D. and Werb, Z. How matrix metalloproteinases regulate cell behavior. Annu Rev Cell Dev Biol 17:463-516; 2001.
95. Brennan, M.; Bhatti, H.; Nerusu, K. C.; Bhagavathula, N.; Kang, S.; Fisher, G. J.; Varani, J. and Voorhees, J. J. Matrix metalloproteinase-1 is the major collagenolytic enzyme responsible for collagen damage in UV-irradiated human skin. Photochem Photobiol 78:43-8; 2003.
96. Berneburg, M.; Grether-Beck, S.; Kurten, V.; Ruzicka, T.; Briviba, K.; Sies, H. and Krutmann, J. Singlet oxygen mediates the UVA-induced generation of the photoaging-associated mitochondrial common deletion. J Biol Chem 274:15345-9; 1999.
97. Valacchi, G.; Rimbach, G.; Saliou, C.; Weber, S. U. and Packer, L. Effect of benzoyl peroxide on antioxidant status, NF-kappaB activity and interleukin-1alpha gene expression in human keratinocytes. Toxicology 165:225-34; 2001.
98. Shindo, Y.; Witt, E. and Packer, L. Antioxidant defense mechanisms in murine epidermis and dermis and their responses to ultraviolet light. J Invest Dermatol 100:260-5; 1993.
99. Shindo, Y.; Witt, E.; Han, D. and Packer, L. Dose-response effects of acute ultraviolet irradiation on antioxidants and molecular markers of oxidation in murine epidermis and dermis. J Invest Dermatol 102:470-5; 1994.
100. Torma, H.; Rollman, 0. and Vahiquist, A. The vitamin A metabolism and expression of retinoid-binding proteins differ in HaCaT cells and normal human keratinocytes. Arch Dermatol Res 291:33945; 1999.
101. Popa, C.; Dicker, A. J.; Dahler, A. L. and Saunders, N. A. Cytochrome P450, CYP26AI, is expressed at low levels in human epidermal keratinocytes and is not retinoic acid-inducible. Br J Dermatol 141:460-8; 1999.
102. Wang, X. D.; Russell, R. M.; Liu, C.; Stickel, F.; Smith, D. E. and Krinsky, N. I. Beta-oxidation in rabbit liver in vitro and in the perfused ferret liver contributes to retinoic acid biosynthesis from beta-apocarotenoic acids. J Biol Chem 271:26490-8; 1996.
103. Bachmann, H.; Desbarats, A.; Pattison, P.; Sedgewick, M.; Riss, G.; Wyss, A.; Cardinault, N.; Duszka, C.; Goralczyk, R. and Grolier, P. Feedback regulation of beta, beta-carotene 15,15′-monooxygenase by retinoic acid in rats and chickens. J Nutr 132:3616-22; 2002.
104. Boulanger, A.; McLemore, P.; Copeland, N. G.; Gilbert, D. J.; Jenkins, N. A.; Yu, S. S.; Gentleman, S. and Redmond, T. M. Identification of beta-carotene 15, 15′-monooxygenase as a peroxisome proliferator-activated receptor target gene. Faseb J 17:1304-6; 2003.
105. Takeda, A.; Morinobu, T.; Takitani, K.; Kimura, M. and Tamai, H. Measurement of retinoids and beta-carotene 15,15′-dioxygenase activity in HR-1 hairless mouse skin with UV exposure. J Nutr Sci Vitaminol (Tokyo) 49:69-72; 2003.
106. Elder, J. T.; Fisher, G. J.; Zhang, Q. Y.; Eisen, D.; Krust, A.; Kastner, P.; Chambon, P. and Voorhees, J. J. Retinoic acid receptor gene expression in human skin. J Invest Dermatol96:425-33; 1991.
107. Wang, Z.; Boudjelal, M.; Kang, S.; Voorhees, J. J. and Fisher, G. J. Ultraviolet irradiation of human skin causes functional vitamin A deficiency, preventable by all-trans retinoic acid pre-treatment. Nat Med 5:418-22.; 1999.
108. Hoffmann, B.; Lehmann, J. M.; Zhang, X. K.; Hermann, T.; Husmann, M.; Graupner, G. and Pfahl, M. A retinoic acid receptor-specific element controls the retinoic acid receptor-beta promoter. Mol Endocrinol 4:1727-36; 1990.
109. Sucov, H. M.; Murakami, K. K. and Evans, R. M. Characterization of an autoregulated response element in the mouse retinoic acid receptor type beta gene. Proc Natl Acad Sci U S A 87:5392-6; 1990.
110. Brand, N. J.; Petkovich, M. and Chambon, P. Characterization of a functional promoter for the human retinoic acid receptor-alpha (hRAR-alpha). Nucleic Acids Res 18:6799-806; 1990.
111. Lehmann, J. M.; Zhang, X. K. and Pfahl, M. RAR gamma 2 expression is regulated through a retinoic acid response element embedded in Sp1 sites. Mol Cell Biol 12:2976-85; 1992.
112. Barger, P. M. and Kelly, D. P. Identification of a retinoid/chicken ovalbumin upstream promoter transcription factor response element in the human retinoid X receptor gamma2 gene promoter. J Biol Chem 272:2722-8; 1997.
113. Guerin, E.; Ludwig, M. G.; Basset, P. and Anglard, P. Stromelysin-3 induction and interstitial collagenase repression by retinoic acid. Therapeutical implication of receptor-selective retinoids dissociating transactivation and AP-1-mediated transrepression. J Biol Chem 272:11088-95; 1997.
114. Pan, L.; Eckhoff, C. and Brinckerhoff, C. E. Suppression of collagenase gene expression by all-trans and 9-cis retinoic acid is ligand dependent and requires both RARs and RXRs. J Cell Biochem 57:575-89; 1995.
115. Lehmann, B. HaCaT cell line as a model system for vitamin D3 metabolism in human skin. J Invest Dermatol 108:78-82; 1997.
116. Tetlow, L. C. and Woolley, D. E. The effects of 1 alpha,25-dihydroxyvitamin D(3) on matrix metalloproteinase and prostaglandin E(2) production by cells of the rheumatoid lesion. Arthritis Res 1:63-70; 1999.

The scope of the present invention is not limited by the description, examples, and suggested uses herein and modifications can be made without departing from the spirit of the invention. Thus, it is intended that the present invention cover modifications and variations of this invention provided that they come within the scope of the appended claims and their equivalents.

1. A method of treating or preventing non-light induced skin aging in an organism comprising the step of administering an effective amount of β-carotene, a precursor of β-carotene, a derivative of β-carotene, a salt of β-carotene, or a combination thereof to an organism in need thereof.

2. A method according to claim 1, wherein the organism is a human.

3. A method according to claim 2, wherein about 1 to about 30 mg per day of the β-carotene, precursor of β-carotene, derivative of β-carotene, salt of β-carotene, or combination thereof is administered.

4. A method according to claim 3, wherein about 5 to about 20 mg per day of the β-carotene, precursor of β-carotene, derivative of β-carotene, salt of β-carotene, or combination thereof is administered.

5. A method according to claim 4, wherein about 10 to about 15 mg per day of the β-carotene, precursor of β-carotene, derivative of β-carotene, salt of β-carotene, or combination thereof is administered.

6. A composition comprising an amount of β-carotene, a derivative of β-carotene, a salt of β-carotene, or a combination thereof effective to treat or prevent of non-light induced skin aging.

7. A composition according to claim 6, wherein the amount of β-carotene, precursor of β-carotene, derivative of β-carotene, salt of β-carotene, or combination thereof present in the composition is from about 1 to about 30 mg.

8. A composition according to claim 7, wherein the amount of β-carotene, precursor of β-carotene, derivative of β-carotene, salt of β-carotene, or combination thereof present in the composition is from about 5 to about 20 mg.

9. A composition according to claim 8, wherein the amount of β-carotene, precursor of β-carotene, derivative of β-carotene, salt of β-carotene, or combination thereof present in the composition is from about 10 to about 15 mg.

10. A composition according to claim 6 in a dosage form selected from the group consisting of a dietary supplement, a food, a feed, a beverage, a fortified food, a fortified feed, a personal care product, a nutraceutical, a functional food, a functional feed, a clinical nutrition product, a food additive, and a feed additive.

11. A method for reducing the basal MMP-10 expression in unirradiated cells of an organism comprising administering an effective amount of β-carotene, a precursor of β-carotene, a derivative of β-carotene, a salt of β-carotene, or a combination thereof to the organism in need thereof.

12. A method according to claim 11, wherein the organism is a human.

13. A method according to claim 12, wherein the β-carotene, precursor of β-carotene, derivative of β-carotene, salt of β-carotene, or combination thereof is administered in an amount from about 1 to about 30 mg per day.

14. A method according to claim 13, wherein the β-carotene, precursor of β-carotene, derivative of β-carotene, salt of β-carotene, or combination thereof is administered in an amount from about 5 to about 20 mg per day.

15. A method according to claim 14, wherein the β-carotene, precursor of β-carotene, derivative of β-carotene, salt of β-carotene, or combination thereof is administered in an amount from about 10 to about 15 mg per day.

16. A method for reducing basal MMP-1 RNA transcription and protein translation in unirradiated cells of an organism comprising administering an effective amount of β-carotene, a precursor of β-carotene, a derivative of β-carotene, a salt of β-carotene, or a combination thereof to the organism in need thereof.

17. A method according to claim 16, wherein the organism is a human.

18. A method according to claim 17, wherein the β-carotene, precursor of β-carotene, derivative of β-carotene, salt of β-carotene, or combination thereof is administered in an amount from about 1 to about 30 mg per day.

19. A method according to claim 18, wherein the β-carotene, precursor of β-carotene, derivative of β-carotene, salt of β-carotene, or combination thereof is administered in an amount from about 5 to about 20 mg per day.

20. A method according to claim 19, wherein the β-carotene, precursor of β-carotene, derivative of β-carotene, salt of β-carotene, or combination thereof is administered in an amount from about 10 to about 15 mg per day.

21. A method for modulating UV-A induced RNA transcription and polypeptide translation of a matrix metalloprotease (MMP) comprising administering to an organism in need thereof an effective amount of a composition comprising β-carotene, a precursor of β-carotene, a derivative of β-carotene, a salt of β-carotene, or a combination thereof.

22. A method according to claim 21, wherein the MMP is selected from the group consisting of MMP-1, MMP-3, MMP-10, and combinations thereof.

23. A method according to claim 22, wherein the MMP is MMP-1 and MMP-10.

24. A method according to claim 21, wherein the modulation comprises a reduction in the MMP RNA or protein levels compared to an organism to which the β-carotene composition is not administered.

25. A method according to claim 21, wherein the organism is a human.

26. A method according to claim 21, wherein the β-carotene, precursor of β-carotene, derivative of β-carotene, salt of β-carotene, or combination thereof is present in the composition in an amount from about 1 to about 30 mg per day.

27. A method according to claim 26, wherein the β-carotene, precursor of β-carotene, derivative of β-carotene, salt of β-carotene, or combination thereof is present in the composition in an amount from about 5 to about 20 mg per day.

28. A method according to claim 27, wherein the β-carotene, precursor of β-carotene, derivative of β-carotene, salt of β-carotene, or combination thereof is present in the composition in an amount from about 10 to about 15 mg per day.

29. A method of treating or ameliorating UVA-induced photoaging comprising administering to an organism in need thereof an effective amount of a composition comprising β-carotene, a precursor of β-carotene, a derivative of β-carotene, a salt of β-carotene, or a combination thereof, which is sufficient to ameliorate the UVA-induced photoaging.

30. A method according to claim 29 wherein the organism is a human.

31. A method according to claim 29, wherein the effective amount is sufficient to reduce the level of MMP RNA transcripts and protein in the skin cells of the organism compared to an organism to which the β-carotene composition is not administered.

32. A method according to claim 31, wherein the MMP RNA transcripts and protein are selected from the group consisting of MMP-1, MMP-3, MMP-10, and combinations thereof.

33. A method according to claim 32, wherein the MMP RNA transcripts and protein are MMP-1 and MMP-10.

34. A method according to claim 29, wherein the β-carotene, precursor of β-carotene, derivative of β-carotene, salt of β-carotene, or combination thereof is present in the composition in an amount from about 1 to about 30 mg per day.

35. A method according to claim 34, wherein the β-carotene, precursor of β-carotene, derivative of β-carotene, salt of β-carotene, or combination thereof is present in the composition in an amount from about 5 to about 20 mg per day.

36. A method according to claim 35, wherein the β-carotene, precursor of β-carotene, derivative of β-carotene, salt of β-carotene, or combination thereof is present in the composition in an amount from about 10 to about 15 mg per day.

37. A composition for modulating the effect of UVA-induced RNA transcription and polypeptide translation of a matrix metalloprotease (MMP) comprising an effective amount of β-carotene, a precursor of β-carotene, a derivative of β-carotene, a salt of β-carotene, or a combination thereof to modulate the transcription and translation of MMPs induced by exposure to UVA.

38. A composition according to claim 37, wherein the amount of β-carotene, precursor of β-carotene, derivative of β-carotene, salt of β-carotene, or combination thereof present in the composition is from about 1 to about 30 mg.

39. A composition according to claim 38, wherein the amount of, β-carotene, precursor of δ-carotene, derivative of , δ-carotene, salt of δ-carotene, or combination thereof present in the composition is from about 5 to about 20 mg per day.

40. A composition according to claim 39, wherein the amount of β-carotene, precursor of β-carotene, derivative of β-carotene, salt of β-carotene, or combination thereof present in the composition is from about 10 to about 15 mg per day.

41. A composition according to claim 37, which is in a dosage form selected from the group consisting of a dietary supplement, a food, a feed, a beverage, a fortified food, a fortified feed, a personal care product, a nutraceutical, a functional food, a functional feed, a clinical nutrition product, a food additive and a feed additive.
